(19) United States
(12) Patent Application Publication
(10) Pub. No.: US 2008/0188478 A1
(43) Pub. Date: Aug. 7, 2008

(54) Compounds Useful In Therapy

(75) Inventors: Justin Stephen Bryans, Kent (GB); Mark Edward Bunnage, Kent (GB); Patrick Stephen Johnson, Kent (GB); Helen Janet Mason, Kent (GB); Lee Richard Roberts, Kent (GB); Thomas Ryckmans, Kent (GB); Alan Stobie, Kent (GB); Toby James Underwood, Kent (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 11/912,592

(22) PCT Filed: Apr. 18, 2006
(86) PCT No.: PCT/IB2006/001071

§ 371 Date: Jan. 18, 2008

Related U.S. Application Data
(60) Provisional application No. 60/674,988, filed on Apr. 26, 2005.

(51) Int. Cl. A61K 31/454; C07D 401/04; C07D 471/04; A61K 31/5025; A61P 25/00; A61K 31/437; C07D 487/04

(52) U.S. Cl. 514/248; 546/200; 546/115; 544/236; 514/322; 514/302

(57) ABSTRACT

Compounds of formula (I),

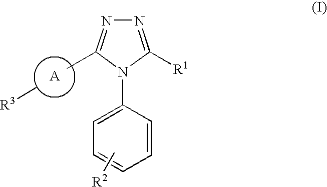

or pharmaceutically acceptable derivatives thereof, wherein:
R¹ represents a group selected from H, CF₃, and C₁₋₆ alkyl (optionally substituted by C₁₋₆ alkyloxy or triazolyl);
R² represents halo;
Ring A represents a 5- or 6-membered heterocyclic ring containing at least one N atom (the ring being optionally bridged with two or more carbon atoms);
   R³ represents a 5- or 6-membered heterocyclic ring containing at least one atom selected from N, O or S, the heterocyclic ring being optionally substituted by one or more groups selected from C₁₋₆ alkyl oxo or NH₂, the heterocyclic ring being further optionally fused to a 5- or 6-membered aryl or heterocyclic ring containing at least one atom selected from N, O or S, the fused aryl or heterocyclic ring being substituted by one or more halo atoms;
   are useful for treating a disorder for which a V1a antagonist is indicated, in particular, dysmenorrhea.

This invention relates to triazole derivatives and to processes for their preparation. It also relates to intermediates used in their preparation, compositions containing them and their uses.

The triazole derivatives of the present invention are vasopressin antagonists. In particular they are antagonists of the V1a receptor and have a number of therapeutic applications, particularly in the treatment of dysmenorrhea (primary and secondary).

There is a high unmet need in the area of menstrual disorders and it is estimated that up to 90% of all menstruating women are affected to some degree. Up to 42% of women miss work or other activities due to menstrual pain and it has been estimated that around 600 million work hours a year are lost in the US as a result {Coco, A. S. (1999). Primary dysmenorrhea. [Review][30 refs]. American Family Physician, 60, 489-96.}.

Menstrual pain in the lower abdomen is caused by myometrial hyperactivity and reduced uterine blood flow. These pathophysiological changes result in abdominal pain that radiates out to the back and legs. This may result in women feeling nauseous, having headaches and suffering from insomnia. This condition is called dysmenorrhea and can be classified as either primary or secondary dysmenorrhea.

Primary dysmenorrhea is diagnosed when no abnormality causing the condition is identified. This affects up to 50% of the female population {Coco, A. S. (1999). Primary dysmenorrhea. [Review][30 refs]. American Family Physician, 60, 489-96; Schroeder, B. & Sanfilippo, J. S. (1999). Dysmenorrhea and pelvic pain in adolescents. [Review][78 refs]. Pediatric Clinics of North America, 46, 555-71}. Where an underlying gynecological disorder is present, such as endometriosis, pelvic inflammatory disease (PID), fibroids or cancers, secondary dysmenorrhea will be diagnosed. Secondary dysmenorrhea is diagnosed in only approximately 25% of women suffering from dysmenorrhea. Dysmenorrhea can occur in conjunction with menorrhagia, which accounts for around 12% of referrals to gynecology outpatients departments.

Currently, women suffering from primary dysmenorrhea are treated with non-steroidal anti-inflammatory drugs (NSAID's) or the oral contraceptive pill. In cases of secondary dysmenorrhea surgery may be undertaken to correct the underlying gynecological disorder.

Women suffering from dysmenorrhea have circulating vasopressin levels which are greater than those observed in healthy women at the same time of the menstrual cycle. Inhibition of the pharmacological actions of vasopressin, at the uterine vasopressin receptor, may prevent dysmenorrhea.

The compounds of the present invention are therefore potentially useful in the treatment of a wide range of disorders, particularly aggression, Alzheimer's disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhea (primary and secondary), emesis (including motion sickness), endometriosis, gastrointestinal disease, glaucoma, gynecological disease, heart disease, intrauterine growth retardation, inflammation (including rheumatoid arthritis), ischemia, ischemic heart disease, lung tumor, micturition disorder, mittlesmerchz, neoplasm, nephrotoxicity, non-insulin dependent diabetes, obesity, obsessive/compulsive disorder, ocular hypertension, preclampsia, premature ejaculation, premature (preterm) labour, pulmonary disease, Raynaud's disease, renal disease, renal failure, male or female sexual dysfunction, septic shock, sleep disorder, spinal cord injury, thrombosis, urogenital tract infection or urolithiasis.

Particularly of interest are the following diseases or disorders:

anxiety, cardiovascular disease (including angina, atherosclerosis, hypertension, heart failure, edema, hypernatremia), dysmenorrhea (primary and secondary), endometriosis, emesis (including motion sickness), intrauterine growth retardation, inflammation (including rheumatoid arthritis), mittlesmerchz, preclampsia, premature ejaculation, premature (preterm) labour and Raynaud's disease.

The compounds of the invention, and their pharmaceutically acceptable salts and solvates, have the advantage that they are selective inhibitors of the V1a receptor (and so are likely to have reduced side effects), they may have a more rapid onset of action, they may be more potent, they may be longer acting, they may have greater bioavailability or they my have other more desirable properties than the compounds of the prior art.

According to the present invention there is provided a compound of formula (I),

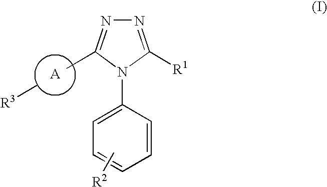

or a pharmaceutically acceptable derivative thereof, wherein:
R¹ represents a group selected from H, CF₃, and C₁₋₆ alkyl (optionally substituted by C₁₋₆ alkyloxy or triazolyl);
R² represents halo;
Ring A represents a 5- or 6-membered heterocyclic ring containing at least one N atom (the ring being optionally bridged with two or more carbon atoms);
R³ represents a 5- or 6-membered heterocyclic ring containing at least one atom selected from N, O or S, the heterocyclic ring being optionally substituted by one or more groups selected from C₁₋₆ alkyl, oxo or NH₂, the heterocyclic ring being further optionally fused to a 5- or 6-membered aryl or heterocyclic ring containing at least one atom selected from N, O or S, the fused aryl or heterocyclic ring being substituted by one or more halo atoms.

In the above definitions, halo means fluoro, chloro, bromo or iodo. Alkyl, alkylene and alkyloxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkyloxy include methoxy, ethoxy, n-propoxy, i-propoxy, n-butoxy, 1-butoxy, sec-butoxy and t-butoxy. Examples of alkylene include methylene, 1,1-ethylene, 1,2-ethylene, 1,1-propylene, 1,2-propylene, 1,3-propylene and 2,2-propylene. Het represents a heterocyclic group, examples of which include tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, 3,4-dihydro-2H-pyranyl, 5,6-dihydro-2H-pyranyl, 2H-pyranyl, 1,2,3,4-tetrahydropyridinyl, 1,2,5,6-tetrahydropyridinyl, pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl and pyrazinyl.

Preferably R¹ represents methyl, CF₃, CH₂OCH₃, or triazolyl-methyl. Preferably R² represents chloro. Preferably ring A represents piperidinyl or piperazinyl. Preferably R³ represents a 5- or 6-membered heterocyclic ring containing at least one atom selected from N, O or S, the heterocyclic ring being optionally substituted by one or more groups selected from C₁₋₆ alkyl, oxo or NH₂, the heterocyclic ring being fused to a 5- or 6-membered aryl or heterocyclic ring containing at least one atom selected from N, O or S, the fused aryl or heterocyclic ring being substituted by one or more halo atoms. More preferably R³ represents a 5- or 6-membered heterocyclic ring containing at least one atom selected from N, O or S, the heterocyclic ring being optionally substituted by one or more groups selected from C₁₋₆ alkyl, oxo or NH₂, the heterocyclic ring being fused to a phenyl or pyridyl ring, the phenyl or pyridyl ring being substituted by one or more halo atoms. Either heterocyclic ring may be aromatic or either may be non-aromatic. An embodiment of the present invention is envisaged wherein R³ comprises fused heterocyclic rings which are both are aromatic. An alternative embodiment of the present invention is envisaged wherein R³ comprises fused heterocyclic rings one of which is aromatic and the other of which is non-aromatic. An alternative embodiment is envisaged wherein R³ comprises fused heterocyclic rings wherein neither heterocyclic ring is aromatic. An alternative embodiment is envisaged wherein R³ comprises an aromatic heterocyclic ring fused to an aryl ring. An alternative embodiment is envisaged wherein R³ comprises a non-aromatic heterocyclic ring fused to an aryl ring. Preferably R³ contains more than three hetero atoms. An embodiment of the present invention is envisaged wherein R³ contains four hetero atoms. Preferred embodiments of the present invention are wherein R³ represents:

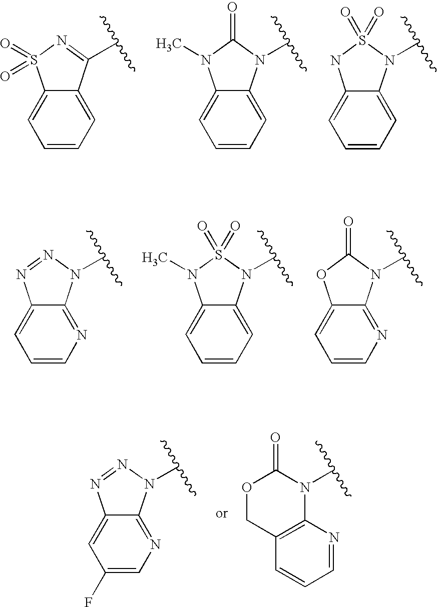

The above described embodiments of the invention may be combined with one or more further embodiments such that further embodiments are provided wherein two or more variables are defined more specifically in combination. For example, within the scope of the invention is a further embodiment wherein the variables R¹, R² and R³ all have the more limited definitions assigned to them in the more specific embodiments described above. All such combinations of the more specific embodiments described and defined above are within the scope of the invention Specific preferred compounds according to the invention are those listed in the Examples section below, and the pharmaceutically acceptable salts or solvates thereof. In particular:
1-{1-[4-(4-chlorophenyl)-5-methyl-4H-1,2,4-triazol-3-yl]piperidin-4-yl}-2-methyl-1H-benzimidazole;
1-{1-[4-(4-chlorophenyl)-5-methyl-4H-1,2,4-triazol-3-yl]piperidin-4-yl}-1,3-dihydro-2H-benzimidazol-2-one;
1-{1-[4-(4-chlorophenyl)-5-methyl-4H-1,2,4-triazol-3-yl]piperidin-4-yl}-3-methyl-1,3-dihydro-2H-benzimidazol-2-one;
5-chloro-1-{1-[4-(4-chlorophenyl)-5-methyl-4H-1,2,4-triazol-3-yl]piperidin-4-yl}-1,3-dihydro-2H-benzimidazol-2-one;
1-{1-[4-(4-chlorophenyl)-5-methyl-4H-1,2,4-triazol-3-yl]piperidin-4-yl}-5-fluoro-1,3-dihydro-2H-benzimidazol-2-one;
1-{1-[4-(4-chlorophenyl)-5-methyl-4H-1,2,4-triazol-3-yl]piperidin-4-yl}-1H-1,2,3-benzotriazole;
3-{1-[4-(4-chlorophenyl)-5-methyl-4H-1,2,4-triazol-3-yl]piperidin-4-yl}[1,3]oxazolo[4,5-b]pyridin-2(3H)-one;
3-{1-[4-(4-chlorophenyl)-5-(2H-1,2,3-triazol-2-ylmethyl)-4H-1,2,4-triazol-3-yl]piperidin-4-yl}[1,3]oxazolo[4,5-b]pyridin-2(3H)-one;
1-[4-(4-chlorophenyl)-5-methyl-4H-1,2,4-triazol-3-yl]-4-(3-isopropyl-1,2,4-oxadiazol-5-yl)piperidine;
4-{1-[4-(4-chlorophenyl)-5-methyl-4H-1,2,4-triazol-3-yl]piperidin-4-yl}-2H-pyrido[3,2-b][1,4]oxazin-3(4H)-one;
1-[4-(4-chlorophenyl)-5-methyl-4H-1,2,4-triazol-3-yl]-4-(5-isopropyl-1,2,4-oxadiazol-3-yl)piperidine;
1-[4-(4-chlorophenyl)-5-(2H-1,2,3-triazol-2-ylmethyl)-4H-1,2,4-triazol-3-yl]-4-(5-isopropyl-1,2,4-oxadiazol-3-yl)piperidine;
3-{1-[4-(4-Chlorophenyl)-5-methyl-4H-1,2,4-triazol-3-yl]piperidin-4-yl}[1,2,4]triazolo[4,3-b]pyridazine;
3-{1-[4-(4-Chlorophenyl)-5-methyl-4H-1,2,4-triazol-3-yl]piperidin-4-yl}-1,3-dihydro-2H-imidazo[4,5-b]pyridin-2-one;
3-{1-[4-(4-Chlorophenyl)-5-methyl-4H-1,2,4-triazol-3-yl]piperidin-4-yl}-3H-imidazo[4,5-b]pyridine;
3-{1-[4-(4-Chlorophenyl)-5-methyl-4H-1,2,4-triazol-3-yl]piperidin-4-yl}-3H-[1,2,3]triazolo[4,5-b]pyridine;
1-{1-[4-(4-Chlorophenyl)-5-methyl-4H-1,2,4-triazol-3-yl]piperidin-4-yl}-1H-benzimidazol-2-amine;
1-{1-[4-(4-Chlorophenyl)-5-methyl-4H-1,2,4-triazol-3-yl]piperidin-4-yl}-3-methyl-1,3-dihydro-2,1,3-benzothiadiazole 2,2-dioxide;
3-{1-[4-(4-Chlorophenyl)-5-methyl)-4H-1,2,4-triazol-3-yl]piperidin-4-yl}-6-fluoro-3H-[1,2,3]triazolo[4,5-b]pyridine;
3-{4-[4-(4-Chlorophenyl)-5-methyl-4H-1,2,4-triazol-3-yl]piperazin-1-yl}-1,2-benzisothiazole;
3-{4-[4-(4-Chlorophenyl)-5-methyl-4H-1,2,4-triazol-3-yl]piperazin-1-yl}-1,2-benzisothiazole 1,1-dioxide;
3-{4-[4-(4-Chlorophenyl)-5-(trifluoromethyl)-4H-1,2,4-triazol-3-yl]piperazin-1-yl}-1,2-benzisothiazole;
3-{4-[4-(4-Chlorophenyl)-5-(trifluoromethyl)-4H-1,2,4-triazol-3-yl]piperazin-1-yl}-1,2-benzisothiazole 1,1-dioxide;
3-{(3-endo)-8-[4-(4-Chlorophenyl)-5-methyl-4H-1,2,4-triazol-3-yl]-8-azabicyclo[3.2.1]oct-3-yl}-2-methyl-3H-imidazo[4,5-c]pyridine;
3-{4-[4-(4-Chlorophenyl)-5-methyl-4H-1,2,4-triazol-3-yl]piperidin-1-yl}-1,2-benzisothiazole;
3-{4-[4-(4-Chlorophenyl)-5-(methoxymethyl)-4H-1,2,4-triazol-3-yl]piperidin-1-yl}-1,2-benzisothiazole;
3-{4-[4-(4-Chlorophenyl)-5-methyl-4H-1,2,4-triazol-3-yl]piperidin-1-yl}isothiazolo[5,4-b]pyridine;
3-{4-[4-(4-Chlorophenyl)-5-methyl-4H-1,2,4-triazol-3-yl]piperidin-1-yl}-1,2-benzisothiazole 1,1-dioxide;
3-{4-[4-(4-Chlorophenyl)-5-methyl-4H-1,2,4-triazol-3-yl]piperidin-1-yl}isoxazolo[4,5-b]pyridine;
   and pharmaceutically acceptable derivatives thereof.

Pharmaceutically acceptable derivatives of the compounds of formula (I) according to the invention include salts, solvates, complexes, polymorphs, prodrugs, stereoisomers, geometric isomers, tautomeric forms, and isotopic variations of compounds of formula (I). Preferably, pharmaceutically acceptable derivatives of compounds of formula (I) comprise salts, solvates, esters and amides of the compounds of formula (I). More preferably, pharmaceutically acceptable derivatives of compounds of formula (I) are salts and solvates.

The pharmaceutically acceptable salts of the compounds of formula (I) include the acid addition and base salts thereof.

Suitable acid addition salts are formed from acids which form non-toxic salts. Examples include the acetate, aspartate, benzoate, besylate, bicarbonate/carbonate, bisulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, D- and L-lactate, malate, maleate, malonate, mesylate, methylsulphate, naphthylate, 2-napsylate, nicotinate, nitrate, orotate, oxalate, palmitate, palmoate, phosphate, hydrogen phosphate, dihydrogen phosphate, saccharate, stearate, succinate, sulphate, D- and L-tartrate, tosylate and trifluoroacetate salts. A particularly suitable salt is the besylate derivative of the compounds of the present invention.

Suitable base salts are formed from bases, which form non-toxic salts. Examples include the aluminium, arginine, benzathine, calcium, choline, diethylamine, diolamine, glycine, lysine, magnesium, meglumine, olamine, potassium, sodium, tromethamine and zinc salts. For a review on suitable salts see Stahl and Wermuth, Handbook of Pharmaceutical Salts: Properties, Selection and Use, Wiley-VCH, Weinheim, Germany (2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention may exist in both unsolvated and solvated forms. The term “solvate” is used herein to describe a molecular complex comprising the compound of the invention and one or more pharmaceutically acceptable solvent molecules, for example, ethanol. The term “hydrate” is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components what may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (I) and pharmaceutically acceptable derivatives include references to salts, solvates and complexes thereof and to solvates and complexes of salts thereof.

The compounds of the invention include compounds of formula (I) as hereinbefore defined, polymorphs, prodrugs, and isomers thereof (including optical, geometric and tautomeric isomers) as hereinafter defined and isotopically-labelled compounds of formula (I).

As stated, the invention includes all polymorphs of the compounds of formula (I) as hereinbefore defined.

Also within the scope of the invention are so-called “prodrugs” of the compounds of formula (I). Thus certain derivatives of compounds of formula (I) which may have little or no pharmacological activity themselves can, when administered into or onto the body, be converted into compounds of formula (I) having the desired activity, for example, hydrolytic cleavage. Such derivatives are referred to as “prodrugs”. Further information on the use of prodrugs may be found in “Pro-drugs as Novel Delivery Systems, Vol. 14, ACS Symposium Series (T Higuchi and W Stella) and “Bioreversible Carriers in Drug Design”, Pergamon Press, 1987 (ed. E B Roche, American Pharmaceutical Association).

Prodrugs in accordance with the invention can, for example, be produced by replacing appropriate functionalities present in the compounds of formula (I) with certain moieties know to those skilled in the art as “pro-moieties” as described, for example, in “Design of Prodrugs” by H Bundgaard (Elsevier, 1985).

Some examples of prodrugs in accordance with the invention include:
(i) where the compound of formula (I) contains a carboxylic acid functionality (—COOH), an ester thereof, for example, replacement of the hydrogen with C₁₋₈ alkyl;
(ii) where the compound of formula (I) contains an alcohol functionality (—OH), an ether thereof, for example, replacement of the hydrogen with C₁₋₆ alkanoyloxymethyl; and
(iii) where the compound of formula (I) contains a primary or secondary amino functionality (—NH₂ or —NHR where R≠H), an amide thereof, for example, replacement of one or both hydrogens with C₁₋₁₀ alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible, and where the compound contains, for example, a keto or oxime group or an aromatic moiety, tautomeric isomerism (‘tautomerism’) may occur. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counter ion is optically active, for example, D-lactate or L-lysine, or racemic, for example, DL-tartrate or DL-arginine.

Cis/trans isomers may be separated by conventional techniques well known to those skilled in the art, for example, fractional crystallisation and chromatography.

Conventional techniques for the preparation/isolation of individual enantiomers include chiral synthesis from a suitable optically pure precursor or resolution of the racemate (or the racemate of a salt or derivative) using, for example, chiral HPLC.

Alternatively, the racemate (or racemic precursor) may be reacted with a suitable optically active compound, for example, an alcohol, or, in the case where the compounds of formula (I) contains an acidic or basic moiety, an acid or base such as tartaric acid or 1-phenylethylamine. The resulting diastereomeric mixture may be separated by chromatography and/or fractional crystallisation and one or both of the diastereomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention also includes all pharmaceutically acceptable isotopic variations of a compound of the formula (I) one or more atoms is replaced by atoms having the same atomic number, but an atomic mass or mass number different from the atomic mass or mass number usually found in nature.

Examples of isotopes suitable for inclusion in the compounds of the invention include isotopes of hydrogen such as 2H and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulphur such as ³⁵S, fluorine such as ¹⁸F, iodine such as ²³I and ¹²⁵I, and chlorine such as ³⁶Cl.

Certain isotopically-labelled compounds of formula (I), for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using appropriate isotopically-labelled reagents in place of the non-labelled reagent previously employed.

Pharmaceutically acceptable solvates in accordance with the invention include those wherein the solvent of crystallisation may be isotopically substituted, e.g. D₂O, d₆-acetone and d₆-DMSO.

The compounds of the invention are useful in therapy. Therefore, a further aspect of the invention is the use of a compound of formula (I), or a pharmaceutically salt or solvate thereof, as a medicament.

The compounds of the invention show activity as V1a antagonists. In particular they are useful in the treatment of a number of conditions including aggression, Alzheimer's disease, anorexia nervosa, anxiety, anxiety disorder, asthma, atherosclerosis, autism, cardiovascular disease (including angina, atherosclerosis, hypertension, heart failure, edema, hypernatremia), cataract, central nervous system disease, cerebrovascular ischemia, cirrhosis, cognitive disorder, Cushing's disease, depression, diabetes mellitus, dysmenorrhea (primary and secondary), emesis (including motion sickness), endometriosis, gastrointestinal disease, glaucoma, gynecological disease, heart disease, intrauterine growth retardation, inflammation (including rheumatoid arthritis), ischemia, ischemic heart disease, lung tumor, micturition disorder, mittlesmerchz, neoplasm, nephrotoxicity, non-insulin dependent diabetes, obesity, obsessive/compulsive disorder, ocular hypertension, preclampsia, premature ejaculation, premature (preterm) labor, pulmonary disease, Raynaud's disease, renal disease, renal failure, male or female sexual dysfunction, septic shock, sleep disorder, spinal cord injury, thrombosis, urogenital tract infection or urolithiasis sleep disorder, spinal cord injury, thrombosis, urogenital tract infection, urolithiasis. Particularly of interest is dysmenorrhea (primary or secondary), more particularly, primary dysmenorrhea.

Therefore, a further aspect of the invention is the method of treatment of a mammal, including a human being, to treat a disorder for which a V1a antagonist is indicated, comprising administering a therapeutically effective amount of a compound of formula (I), or a pharmaceutically acceptable salt or solvate thereof, to the mammal. In particular, the compounds of formula (I) are useful in treating anxiety, cardiovascular disease (including angina, atherosclerosis, hypertension, heart failure, edema, hypernatremia), dysmenorrhea (primary and secondary), endometriosis, emesis (including motion sickness), intrauterine growth retardation, inflammation (including rheumatoid arthritis), mittlesmerchz, preclampsia, premature ejaculation, premature (preterm) labour or Raynaud's disease. Even more particularly, they are useful in treating dysmenorrhea (primary or secondary).

A further aspect of the present invention is the use of a compound of formula (I), or a pharmaceutically acceptable salt or solvate thereof, in the manufacture of a medicament for the treatment of a disorder for which a V1a receptor antagonist is indicated.

All of the compounds of the formula (I) can be prepared by the procedures described in the general methods presented below or by the specific methods described in the Examples section and the Preparations section, or by routine modifications thereof. The present invention also encompasses any or one or more of these processes for preparing the compounds of formula (I), in addition to any novel intermediates used therein.

Unless otherwise provided herein:
WSCDI means 1-(3-dimethylaminopropyl)-3-ethylcarbodiimide hydrochloride;
DCC means N,N′-dicyclohexylcarbodiimide;
HOAT means 1-hydroxy-7-azabenzotriazole;
HOBT means 1-hydroxybenzotriazole hydrate;
PyBOP® means Benzotriazol-1-yloxytris(pyrrolidino)phosphoniumhexa fluorophosphate;
PyBrOP® means bromo-tris-pyrrolidino-phosphoniumhexafluoro phosphate;
HBTU means O-benzotriazol-1-yl-N,N,N′,N′-tetramethyluronium hexafluoro phosphate;
mCPBA means meta-chloroperbenzoic acid;
Et₃N means triethylamine;
NMM means N-methylmorpholine;
Boc means tert-butoxycarbonyl;
CBz means benzyloxycarbonyl;
p-TSA means p-toluenesulphonic acid;
DBU means 1,8-Diazabicyclo[5.4.0]undec-7-ene;
MeI means methyl iodide;
MeTosylate means methyl p-toluenesulphonate;
MeOH means methanol, EtOH means ethanol, and EtOAc means ethyl acetate; MeCN means acetonitrile,
THF means tetrahydrofuran, DMSO means dimethyl sulphoxide, and DCM means dichloromethane, DMF means N,N-dimethylformamide, NMP means N-methyl-2-pyrrolidinone, DMA means dimethylacetamide;
AcOH means acetic acid, TFA means trifluoroacetic acid;
Me means methyl, Et means ethyl;
Cl means chloro; and
OH means hydroxy.

In the following general methods, R¹, R², R³, and ring A, are as previously defined for a compound of the formula (I) unless otherwise stated.

When R³ is attached to a nitrogen atom within ring A, then compounds of formula (I) may be prepared according to Scheme 1.

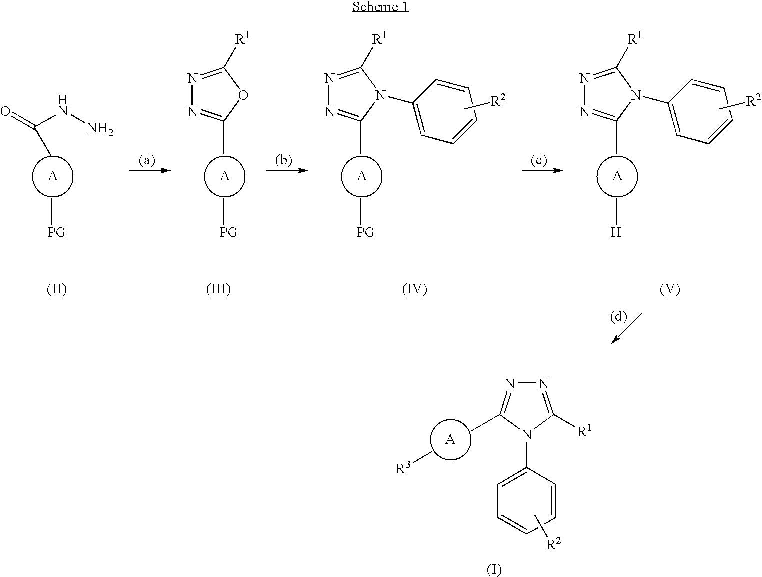

PG represents a suitable N protecting group, typically a benzyl, BOC or CBz group, and preferably BOC.

Compounds of formula (II) may be obtained as described in WO 97/03986, or by reaction of the corresponding lower alkyl ester (e.g. methyl or ethyl) with hydrazine under standard conditions.

Step (a): The compounds of formula (III) may be prepared by reaction of the hydrazide of formula (II) with a suitable acetal (e.g. N,N-dimethylformamide dimethyl acetal), in a suitable solvent, such as THF or DMF, at between room temperature and about 60° C., for up to 18 hours. The resulting intermediate may then be treated under acid catalysis (e.g. p-TSA or TFA) in a high boiling point solvent (e.g. toluene or xylene) for about 18 hours, to provide the compound of formula (III). Preferred conditions: 1.5 eq. of acetal (e.g. N,N-dimethylformamide dimethyl acetal), in THF at room temperature to 60° C., for about 18 hours, followed by p-TSA (cat.) in toluene at reflux for 18 hours.

Step (b): Formation of the triazole (IV) may be achieved by reaction of the compound of formula (III) with a suitable aniline, in the presence of a suitable acid catalyst, such as TFA or p-TSA, in a suitable high boiling solvent (e.g. toluene or xylene), at an elevated temperature.

Preferred conditions: 1 eq. (III), 0.8 eq. TFA, 1.2 eq. aniline in toluene at about the reflux temperature for up to 18 hours.

Step (c): Deprotection of compound (1V) is undertaken using standard methodology, as described in “Protecting Groups in Organic Synthesis” by T. W. Greene and P. Wutz.

Preferred conditions when PG represents BOC: 1 eq. (IV), excess 4M HCl in dioxan in MeOH, dioxan or DCM at about room temperature for up to 18 hours.

Alternatively, when PG represents BOC, compounds of formula (V) may be prepared directly from compounds of formula (III) by treatment with an excess of TFA, (typically 1.7 to 3.5 eq.) and the appropriate aniline, in toluene at the reflux temperature of the reaction, for up to 2 days.

Step (d): The compound of formula (I) may be prepared by alkylation of the compound of formula (V) using an appropriate alkylating agent, R³LG, (where LG represents a leaving group, typically a halo atom, and preferably Cl) in the presence of a suitable base (e.g. K₂CO₃, Cs₂CO₃, DBU) in a suitable solvent (e.g. MeCN, DMF) at between room temperature and the reflux temperature of the reaction.

Preferred conditions are: 1 eq. (V), 1.2 eq. R³LG, 1.0-1.1 eq. DBU in MeCN at room temperature for up to 48 hours.

Compounds of formula (I), when R³ is attached to a N atom within ring A, and when ring A is attached to the triazole through a N atom, may alternatively be prepared as described in Scheme 2.

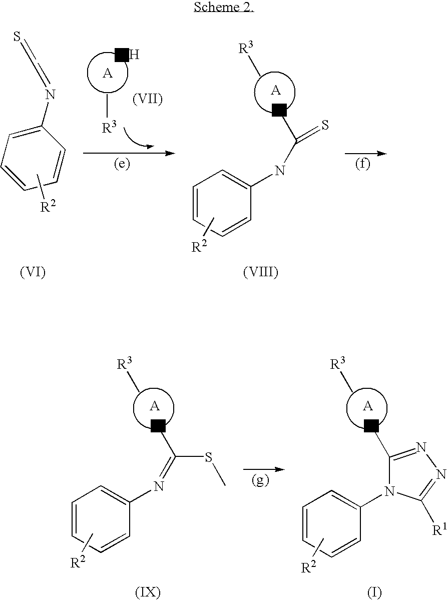

Compounds of formula (VI) are available commercially.

Compounds of formula (VII) may be obtained by analogy with methods described in the literature e.g. Henning et al J. Med. Chem. 1987; 30; 8140819, Butler et al WO 02/20011, Armour et al WO 00/39125 or Cumming et al WO 04/099178.

Step (e): Compounds of formula (VIII) may be prepared by reaction of approximately equimolar amounts of the isothiocyanate of formula (VI) and the amine, or amine salt, of formula (VII) in a suitable solvent (e.g. EtOH, DCM), optionally in the presence of a base (e.g. Et₃N, Hünig's base) at room temperature for between 2 and 48 hours.

Preferred conditions: 1.0 eq. (VI), 1.0 eq. (VII), optionally in the presence of 1.3 eq. Et₃N in EtOH or DCM at room temperature for 2 hours.

Step (f): Compounds of formula (IX) may be prepared by methylation of the thiourea of formula (VIII) using a suitable methylating agent (e.g. MeI, MeTosylate), in the presence of a suitable base (e.g. KOt-Bu) in a suitable solvent (e.g. THF, ether) at between 0° C. and the reflux temperature of the reaction for about 18 hours.

Preferred conditions: 1 eq (VIII), 1-1.2 eq. KOt-Bu, 1.0 eq. MeTosylate, in THF at room temperature for up to 18 hours.

Step (g): Compounds of formula (I) may be prepared by reaction of compounds of formula (IX) with a suitable hydrazide (R¹CONHNH₂) optionally under acidic catalysis (e.g. TFA, p-TSA) in a suitable solvent (e.g. THF, n-BuOH) at between room temperature and the reflux temperature of the reaction.

Preferred conditions: 1 eq. (IX), 0.5-1.5 eq. TFA, 1.0-3.0 eq. of hydrazide (R¹CONHNH₂) in THF at reflux for up to 3 hours.

Alternatively, compounds of formula (I) may be prepared from compounds of formula (VIII), via the compound of formula (IX) in a “one-pot” procedure.

Certain compounds of formula (I), where R³ represents a 5- or 6-membered heterocyclic ring fused to a 5- or 6-membered aryl or heterocyclic ring, may be prepared as shown in Scheme 3 below.

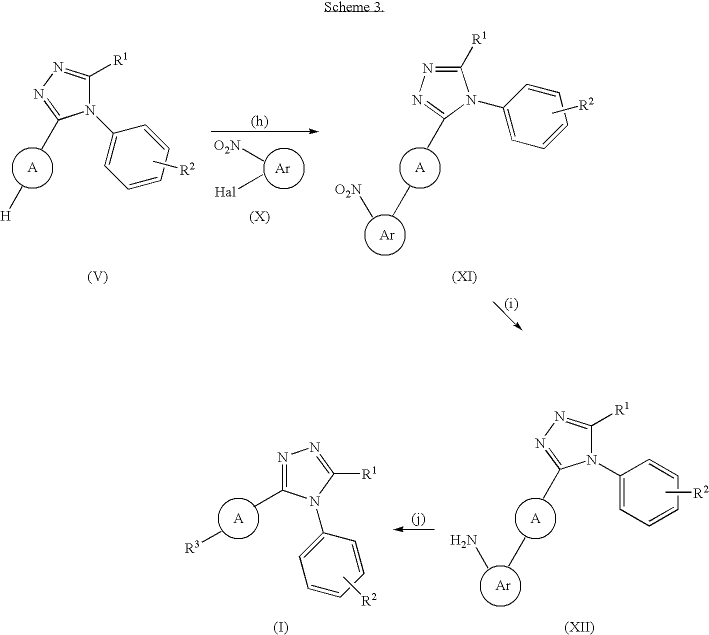

Ring Ar represents an aryl or heterocyclic 5- or 6-membered ring.

Hal represents halide, typically fluoro, chloro or bromo, and preferably fluoro or chloro.

Step (h): Compounds of formula (X) may be prepared by reaction of the amine of formula (V) with an appropriate halide of formula (X), optionally in the presence of a suitable base (e.g. Et₃N, Hünig's base, NMM) in a suitable solvent (e.g. THF, DMF) at between room temperature and the reflux temperature of the reaction.

Preferred conditions are: 1 eq. amine (V), 1 eq. (X), 0-3 eq. Et₃N, in THF or THF/DMF at between room temperature and the reflux temperature of the reaction for about 24 hours.

Step (i): The compound of formula (XII) may be prepared by reduction of the compound of formula (XI) under suitable reducing conditions. Typically this may be achieved by hydrogenation using a suitable catalyst (e.g. Raney® Ni) in a suitable solvent such as EtOH, MeOH or THF at about room temperature, or in the presence of a reducing metal system (e.g. SnCl₂/HCl) in a solvent such as ethanol, at elevated temperature.

Preferred conditions are: Raney® Ni, nitro compound (XI), in EtOH and THF at 30 psi H₂ at room temperature for about 18 hours.

Step (j): The compounds of formula (I) may be obtained by standard methodology, for example those as described in Comprehensive Heterocyclic Chemistry, Katritzky et al, published by Pergamon, N.Y., or by the methods described below:

Where R³ represents:

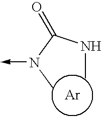

then preferred conditions are: 1 eq. (XII), 3 eq. CDI, in THF at between room temperature and the reflux temperature of the reaction, for about 25 hours.

Where R³ represents:

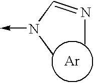

preferred conditions are: 1 eq. (XII) in formic acid at reflux for about 18 hours.

Where R³ represents:

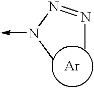

preferred conditions are: 1 eq. (XII), 1.05 to 1.1 eq. NaNO₂, in HCl (aq) at 0° C. for up to 1 hour.

Where R³ represents:

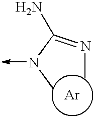

preferred conditions are: 1 eq. (XII), 1.4 eq. BrCN, in THF at reflux for up to 66 hours.

Where R³ represents

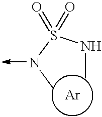

preferred conditions are: 1 eq. (XII), 2 eq. H₂NSO₂NH₂ in pyridine at reflux for about 18 hours.

Alternatively compounds of formula (I), where R³ represents

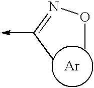

may be prepared as shown in Scheme 4 below.

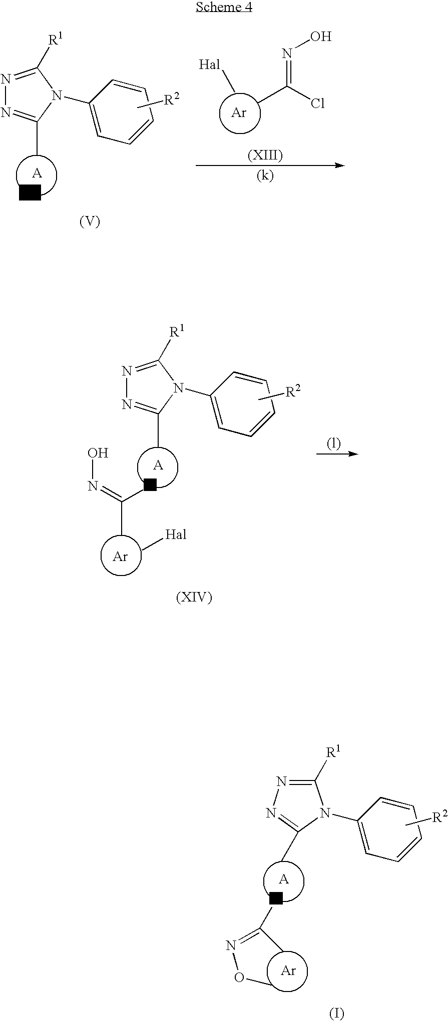

Hal represents a halogen, typically For Cl and preferably F.

Step (k): The compound of formula (XIV) may be prepared by reaction of the piperidine of formula (V) with an imidoyl chloride of formula (XIII) in the presence of a suitable base, typically Et₃N, NMM or Hünig's base, in a suitable solvent, such as DCM, MeCN at about room temperature for about 18 hours.

Preferred conditions: 1 eq. (XIII), 1.5 eq. (V), 3 eq. Et₃N in DCM at room temperature for 18 hours. The compounds of formula (XIII) may be prepared by analogy with the methods of Liu et al J. Org. Chem. 1980; 45; 3916-3918 or Lam et al Biiorg. Med. Chem. Lett. 13(10); 1795; 2003.

Step (l): The compound of formula (I) may be obtained by cyclisation of the compound of formula (XIV). This reaction may be achieved by treatment with a suitable base (e.g. K₂CO₃, NaH) in a suitable solvent or mixture of solvents (e.g. toluene, THF, DMF) at an elevated temperature for about 24 hours, followed by treatment with a suitable acidic alcohol solution. (e.g. AcOH/EtOH). Preferred conditions: 1 eq. (XIV), 1.1 eq. NaH, in toluene for 18 hours at reflux, followed by AcOH in EtOH.

Compounds of formula (III), where R¹ represents C₁ alkyl substituted by C₁-C₆ alkyloxy, may alternatively be prepared as shown in Scheme 5, below.

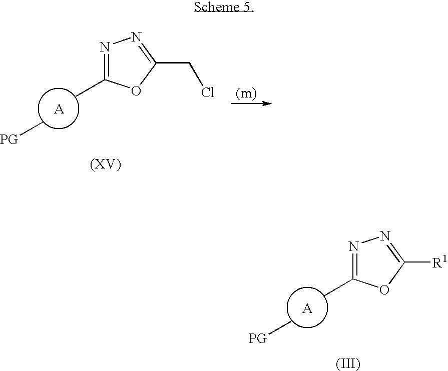

Step (m): Compounds of formula (III) may be prepared by reaction of the compound of formula (XV) with a suitable alcohol, in the presence of a suitable base (e.g. KOt-Bu, NaH) in a suitable solvent (e.g. DMF, MeCN or R¹⁰H) at between room temperature and the reflux temperature of the reaction for about 18 hours.

Preferred conditions: 1 eq. (XV), 1.5 eq. KOt-Bu, in R¹⁰H at between room temperature and 50° C. for up to 18 hours.

Compounds of formula (III) where Q represents NR³, or Q represents a direct link and is attached to a N atom within ring A, may alternatively be prepared as shown in Scheme 6.

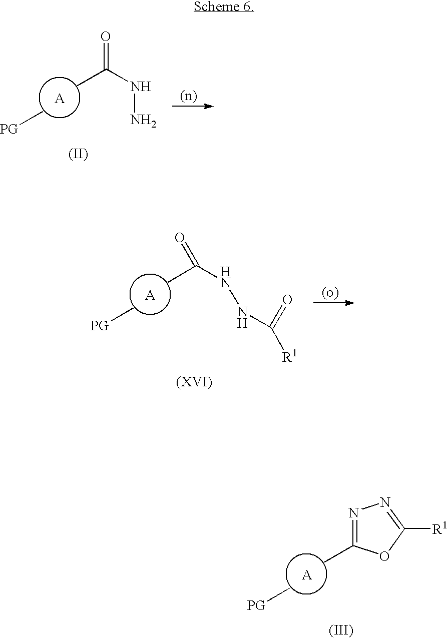

Step (n): The di-acylhydrazides of formula (XVI) may be prepared by coupling of the hydrazides of formula (II) with the acid or acid chloride (R¹COT, where T represents Cl or OH), using standard methodology for reacting an acid or acid chloride with an amine.

Step (o): The oxadiazole of formula (III) may be prepared by cyclisation of the compound of formula (XVI), typically under acid catalysis (e.g. polyphosphoric acid, POCl₃, triflic anhydride/pyridine or 1-methylimidazole), optionally in a suitable solvent (e.g. DCM) at between 0° C. and the reflux temperature of the reaction.

It will be appreciated by those skilled in the art, that certain compounds of formula (I) may undergo standard reactions (e.g. alkylation) or functional group transformations (e.g. oxidation) to provide alternative compounds of formula (I). This is exemplified by the Examples 18 and 28, below.

Compounds of the invention intended for pharmaceutical use may be administered as crystalline or amorphous products. They may be obtained, for example, as solid plugs, powders, or films by methods such as precipitation, crystallisation, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

A further aspect of the invention is a pharmaceutical formulation including a compound of formula (I), or a pharmaceutically acceptable salt or solvate thereof, together with a pharmaceutically acceptable excipient, diluent or carrier. In a further embodiment there is provided the pharmaceutical formulation for administration either prophylactically or when pain commences.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve, which delivers a metered amount. The overall daily dose will typically be in the range 0.01 μg to 15 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.01 mg to 15 mg depending, of course, on the mode of administration. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The compounds of the present invention may be tested in the screens set out below:

1.0 V_{1A} Filter Binding Assay

1.1 Membrane Preparation

Receptor binding assays were performed on cellular membranes prepared from CHO cells stably expressing the human V_{1A} receptor, (CHO-hV_{1A}). The CHO-hV_{1A} cell line was kindly provided under a licensing agreement by Marc Thibonnier, Dept. of Medicine, Case Western Reserve University School of Medicine, Cleveland, Ohio. CHO-hV_{1A} cells were routinely maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. For bulk production of cell pellets, adherent CHO-hV_{1A} cells were grown to confluency of 90-100% in 850 cm² roller bottles containing a medium of DMEM/Hams F12 Nutrient Mix supplemented with 10% fetal bovine serum, 2 mM L-glutamine and 15 mM HEPES. Confluent CHO-hV_{1A} cells were washed with phosphate-buffered saline (PBS), harvested into ice cold PBS and centrifuged at 1,000 rpm. Cell pellets were stored at −80° C. until use. When required, the cell pellets were thawed on ice and homogenised in membrane preparation buffer consisting of 50 mM Tris-HCl, pH 7.4, 5 mM MgCl₂ and supplemented with a protease inhibitor cocktail, (Roche). The cell homogenate was centrifuged at 1000 rpm, 10 min, 4° C. and the supernatant was removed and stored on ice. The remaining pellet was homogenised and centrifuged as before. The supernatants were pooled and centrifuged at 25,000×g for 30 min at 4° C. The pellet was resuspended in freezing buffer consisting of 50 mM Tris-HCl, pH 7.4, 5 mM MgCl₂ and 20% glycerol and stored in small aliquots at −80° C. until use. Protein concentration was determined using Bradford reagent and BSA as a standard.

1.2 V_{1A} Filter Binding

Protein linearity, followed by saturation binding studies were performed on each new batch of membrane. A membrane concentration was chosen that gave specific binding on the linear portion of the curve. Saturation binding studies were then performed using various concentrations of [³H]-arginine vasopressin, [³H]-AVP (0.05 nM-100 nM) and the K_{d} and Bₘₐₓ were determined. Compounds were tested for their effects on [³H]-AVP binding to CHO-hV_{1A} membranes, (3H-AVP; specific activity 65.5 Ci/mmol; NEN Life Sciences). The compounds were solubilised in dimethylsulfoxide (DMSO) and diluted to a working concentration of 10% DMSO with assay buffer containing 50 mM Tris-HCL pH 7.4, 5 mM MgCl₂ and 0.05% BSA. 25 μl compound and 25 μl [³H]-AVP, (final concentration at or below K_{d} determined for membrane batch, typically 0.5 nM-0.6 nM) were added to a 96-well round bottom polypropylene plate. The binding reaction was initiated by the addition of 200 μl membrane and the plates were gently shaken for 60 minutes at room temperature. The reaction was terminated by rapid filtration using a Filtermate Cell Harvester (Packard Instruments) through a 96-well GF/B UniFilter Plate which had been presoaked in 0.5% polyethyleneimine to prevent peptide sticking. The filters were washed three times with 1 ml ice cold wash buffer containing 50 mM Tris-HCL pH 7.4 and 5 mM MgCl₂. The plates were dried and 50 μl Microscint-0 (Packard instruments) was added to each well. The plates were sealed and counted on a TopCount Microplate Scintillation Counter (Packard Instruments). Non-specific binding (NSB) was determined using 1 μM unlabelled d(CH2)5Tyr(Me)AVP ([β-mercapto-β,β-cyclopentamethylenepropionyl, 0-Me-Tyr², Arg⁸]-vasopressin) (βMCPVP), (Sigma). The radioligand binding data was analysed using a four parameter logistic equation with the min forced to 0%. The slope was free fitted and fell between −0.75 and −1.25 for valid curves. Specific binding was calculated by subtracting the mean NSB cpm from the mean Total cpm. For test compounds the amount of ligand bound to the receptor was expressed as % bound=(sample cpm−mean NSB cpm)/specific binding cpm×100. The % bound was plotted against the concentration of test compound and a sigmoidal curve was fitted. The inhibitory dissociation constant (Kᵢ) was calculated using the Cheng-Prusoff equation: Kᵢ=IC₅₀/(1+[L]/K_{d}) where [L] is the concentration of ligand present in the well and K_{d} is the dissociation constant of the radioligand obtained from Scatchard plot analysis.

2.0 V_{1A} Functional Assay; Inhibition of AVP/V_{1A}-R Mediated Ca²⁺ Mobilization by FLIPR (Fluorescent Imaging Plate Reader) (Molecular Devices)

Intracellular calcium release was measured in CHO-hV_{1A} cells using FLIPR, which allows the rapid detection of calcium following receptor activation. The CHO-hV_{1A} cell line was kindly provided under a licensing agreement by Marc Thibonnier, Dept. of Medicine, Case Western Reserve University School of Medicine, Cleveland, Ohio. CHO-V_{1A} cells were routinely maintained at 37° C. in a humidified atmosphere with 5% CO₂ in DMEM/Hams F12 nutrient mix supplemented with 10% fetal bovine serum, 2 mM L-glutamine, 15 mM HEPES and 400 μg/ml G418. On the afternoon before the assay cells were plated at a density of 20,000 cells per well into black sterile 96-well plates with clear bottoms to allow cell inspection and fluorescence measurements from the bottom of each well. Wash buffer containing Dulbecco's phosphate buffered saline (DPBS) and 2.5 mM probenecid and loading dye consisting of cell culture medium containing 4 μM Fluo-3-AM (dissolved in DMSO and pluronic acid), (Molecular Probes) and 2.5 mM probenecid was prepared fresh on the day of assay. The compounds were solubilised in DMSO and diluted in assay buffer consisting of DPBS containing 1% DMSO, 0.1% BSA and 2.5 mM probenecid. The cells were incubated with 100 μl loading dye per well for 1 hour at 37° C. in humidified atmosphere with 5% CO₂. After dye loading the cells were washed three times in 100 μl wash buffer using a Denley plate washer. 100 μl wash buffer was left in each well. Intracellular fluorescence was measured using FLIPR. Fluorescence readings were obtained at 2s intervals with 50 μl of the test compound added after 30s. An additional 155 measurements at 2s intervals were then taken to detect any compound agonistic activity. 50 μl of arginine vasopressin (AVP) was then added so that the final assay volume was 200 μl. Further fluorescence readings were collected at 1s intervals for 120s. Responses were measured as peak fluorescence intensity (FI). For pharmacological characterization a basal FI was subtracted from each fluorescence response. For AVP dose response curves, each response was expressed as a % of the response to the highest concentration of AVP in that row. For IC₅₀ determinations, each response was expressed as a % of the response to AVP. IC₅₀ values were converted to a modified K_{b} value using the Cheng-Prusoff equation which takes into account the agonist concentration, [A], the agonist EC₅₀ and the slope: K_{b}=IC₅₀/(2+[A]/A₅₀]n)^{1/n}−1 where [A] is the concentration of AVP, A₅₀ is the EC₅₀ of AVP from the dose response curve and n=slope of the AVP dose response curve.

The compounds of the invention may be administered alone or in combination with one or more other compounds of the invention or in combination with one or more other drugs (or as any combination thereof). The compounds of the present invention may be administered in combination with an oral contraceptive. Thus in a further aspect of the invention, there is provided a pharmaceutical product containing an V1a antagonist and an oral contraceptive as a combined preparation for simultaneous, separate or sequential use in the treatment of dysmenorrhea.

The compounds of the present invention may be administered in combination with a PDE5 inhibitor. Thus in a further aspect of the invention, there is provided a pharmaceutical product containing a V1a antagonist and a PDEV inhibitor as a combined preparation for simultaneous, separate or sequential use in the treatment of dysmenorrhea.

PDEV inhibitors useful for combining with V1a antagonists include, but are not limited to:
(i) 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g. as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methyl piperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethyl piperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethyl piperazin-1-ylsulphonyl)-2-(2-methoxy-1 (R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethyl piperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethyl piperazine (see WO 01/27113, Example 8); 5-[2-iso-Butoxy-5-(4-ethylpiperazin-1-ylsulphonyl)pyridin-3-yl]-3-ethyl-2-(1-methyl piperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351, Clalis®), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propyl imidazo[5,1-f]-as-triazin-2-yl)-4-ethoxyphenyl]sulphonyl]-4-ethylpiperazine, i.e. the compound of examples 20, 19, 337 and 336 of published international application WO99/24433; the compound of example 11 of published international application WO93/07124 (EISAI); compounds 3 and 14 from Rotella D P, J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide.
(ii) 4-bromo-5-(pyridylmethylamino)-6-[3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propyl indole-6-carboxylate; 3-acetyl-1-(2-chlorobenzyl)-2-propyl indole-6-carboxylate; 4-bromo-5-(3-pyridylmethylamino)-6-(3-(4-chlorophenyl)propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

The contents of the published patent applications and journal articles and in particular the general formulae of the therapeutically active compounds of the claims and exemplified compounds therein are incorporated herein in their entirety by reference thereto.

Preferably the PDEV inhibitor is selected from sildenafil, tadalafil, vardenafil, DA-8159 and 5-[2-ethoxy-5-(4-ethyl piperazin-1-ylsulphonyl)pyridin-3-yl]-3-ethyl-2-[2-methoxyethyl]-2,6-dihydro-7H-pyrazolo[4,3-d]pyrimidin-7-one. Most preferably the PDE5 inhibitor is sildenafil and pharmaceutically acceptable salts thereof. Sildenafil citrate is a preferred salt.

The compounds of the present invention may be administered in combination with an NO donor. Thus in a further aspect of the invention, there is provided a pharmaceutical product containing a V1a antagonist and a NO donor as a combined preparation for simultaneous, separate or sequential use in the treatment of dysmenorrhea.

The compounds of the present invention may be administered in combination with L-arginine, or as an arginate salt. Thus in a further aspect of the invention, there is provided a pharmaceutical product containing a V1a antagonist and L-arginine as a combined preparation for simultaneous, separate or sequential use in the treatment of dysmenorrhea.

The compounds of the present invention may be administered in combination with a COX inhibitor. Thus in a further aspect of the invention, there is provided a pharmaceutical product containing a V1a antagonist and a COX inhibitor as a combined preparation for simultaneous, separate or sequential use in the treatment of dysmenorrhea.

COX inhibitors useful for combining with the compounds of the present invention include, but are not limited to:
(i) ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester (See Wenk, et al., Europ. J. Pharmacol. 453:319-324 (2002));
(ii) meloxicam, (CAS registry number 71125-38-7; described in U.S. Pat. No. 4,233,299), or a pharmaceutically acceptable salt or prodrug thereof;
(iii) celecoxib (U.S. Pat. No. 5,466,823), valdecoxib (U.S. Pat. No. 5,633,272), deracoxib (U.S. Pat. No. 5,521,207), rofecoxib (U.S. Pat. No. 5,474,995), etoricoxib (International Patent Application Publication No. WO 98/03484), JTE-522 (Japanese Patent Application Publication No. 9052882), or a pharmaceutically acceptable salt or prodrug thereof;
(iv) Parecoxib (described in U.S. Pat. No. 5,932,598), which is a therapeutically effective prodrug of the tricyclic Cox-2 selective inhibitor valdecoxib (described in U.S. Pat. No. 5,633,272), in particular sodium parecoxib;
(v) ABT-963 (described in International Patent Application Publication No. WO 00/24719)
(vi) Nimesulide (described in U.S. Pat. No. 3,840,597), flosulide (discussed in J. Carter, Exp. Opin. Ther. Patents. 8(1), 21-29 (1997)), NS-398 (disclosed in U.S. Pat. No. 4,885,367), SD 8381 (described in U.S. Pat. No. 6,034,256), BMS-347070 (described in U.S. Pat. No. 6,180,651), S-2474 (described in European Patent Publication No. 595546) and MK-966 (described in U.S. Pat. No. 5,968,974);
   The contents of any of the patent applications, and in particular the general formulae of the therapeutically active compounds of the claims and exemplified compounds therein, are incorporated herein in their entirety by reference thereto.

The invention is illustrated by the Preparations and Examples described below. Where it is stated that compounds were prepared in the manner described for an earlier Preparation or Example, the skilled person will appreciate that reaction times, number of equivalents of reagents and reaction temperatures may be modified for each specific reaction, and that it may nevertheless be necessary or desirable to employ different work-up or purification conditions.

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used for common solvents: CDCl₃, deuterochloroform; D₆-DMSO, deuterodimethylsulphoxide; CD₃OD, deuteromethanol; THF, tetrahydrofuran. “Ammonia” refers to a concentrated solution of ammonia in water possessing a specific gravity of 0.88. Where thin layer chromatography (TLC) has been used it refers to silica gel TLC using silica gel 60 F254 plates, R_{f} is the distance traveled by a compound divided by the distance traveled by the solvent front on a TLC plate. When microwave radiation is employed, the two microwaves used are the Emrys Creator and the Emrys Liberator, both supplied by Personal Chemistry Ltd. The power range is 15-300W at 2.45 GHz. The actual power supplied varies during the course of the reaction in order to maintain a constant temperature.

### Preparation 1

### Ethyl 4-[(4-fluoro-2-nitrophenyl)amino]piperidine-1-carboxylate

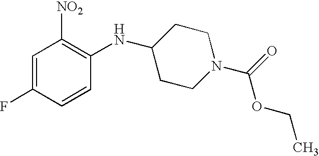

Sodium carbonate (9.12 g, 86 mmol) was added to a solution of 1-chloro-4-fluoro-2-nitrobenzene (15 g, 86 mmol) in cyclohexanol (100 ml). Ethyl 4-amino-piperidine-1-carboxylate (14.68 ml, 86 mmol), followed by potassium iodide (143 mg, 0.86 mmol) was then added, and the reaction mixture was heated at 160° C. for 2 days. The cooled mixture was partitioned between water (300 ml) and toluene (250 ml), the layers were separated, and the aqueous solution was extracted further with toluene (250 ml). The organic layers were combined, washed with brine, dried over magnesium sulfate, filtered and concentrated under reduced pressure. The crude product was purified by column chromatography on silica gel using pentane:ethyl acetate as eluant (80:20 v/v) to yield an orange solid (33 g). This was triturated with diethyl ether and then filtered, to yield the title compound (8 g, 30%).

¹H NMR (400 MHz, CDCl₃): δ 1.27 (t, 3H), 1.53-1.61 (m, 2H), 2.05-2.08 (m, 2H), 3.10 (t, 2H), 3.66 (m, 1H), 4.05-4.09 (m, 2H), 4.15 (q, 2H), 6.85 (dd, 1H), 7.24 (m, 1H), 7.91 (dd, 1H), 7.99 (brs, 1H); LRMS APCI⁺ m/z 312 [MH]⁺.

### Preparation 2

### Ethyl 4-[(2-amino-4-fluorophenyl)amino]piperidine-1-carboxylate

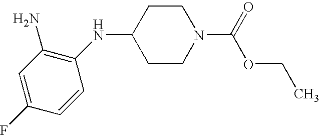

Raney® Nickel (2.5 g) was added to a solution of the nitrobenzene of preparation 1 (8 g, 26 mmol) in ethanol (25 ml) and tetrahydrofuran (50 ml). The reaction mixture was hydrogenated at 30 psi, at room temperature, for 1 hour. The reaction mixture was then filtered through Arbocel®. The filtrate was concentrated under reduced pressure to yield the title compound (4.75 g, 65%) as an oil, which solidified upon standing.

¹H NMR (400 MHz, CDCl₃): δ 1.26 (t, 3H), 1.32-1.40 (m, 2H), 1.97-2.01 (m, 2H), 2.95 (t, 2H), 3.27 (m, 1H), 3.63 (brs, 1H), 4.06-4.16 (m, 4H), 6.41-6.47 (m, 2H), 6.62 (m, 1H); LRMS APCI⁺ m/z 282 [MH]⁺.

### Preparation 3

### Ethyl 4-(5-fluoro-2-oxo-2,3-dihydro-1H-benzimidazol-1-yl)piperidine-1-carboxylate

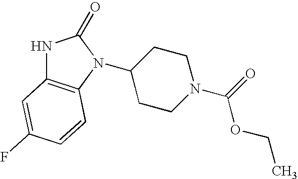

N,N′-Carbonyldiimidazole (6.44 g, 40 mmol) was added to a solution of the aminopiperidine of preparation 2 (4.47 g, 16 mmol) in tetrahydrofuran (100 ml). The reaction mixture was then heated at reflux for 18 hours. Additional N,N′-carbonyldiimidazole (6.44 g, 40 mmol) was added and the reaction mixture was stirred at reflux for a further 2 hours. The cooled reaction mixture was then partitioned between water (100 ml) and ethyl acetate (100 ml), the layers were separated and the aqueous solution was further extracted with ethyl acetate (100 ml). The organic solutions were combined, washed with brine, dried over magnesium sulfate, filtered and concentrated under reduced pressure to provide a gum. This was triturated with diethyl ether, and the solid was filtered to yield the title compound as a pale brown solid.

¹H NMR (400 MHz, CD₃OD): δ 1.28 (t, 3H), 1.80 (d, 2H), 2.29-2.40 (m, 2H), 2.92-3.02 (m, 2H), 4.15 (q, 2H), 4.31 (d, 2H), 4.40 (m, 1H), 6.78-6.84 (m, 2H), 7.19 (m, 1H); LRMS APCI⁺ m/z 308 [MH]⁺.

### Preparation 4

### 5-Fluoro-1-piperidin-4-yl-1,3-dihydro-2H-benzimidazol-2-one

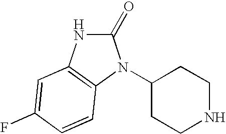

The piperidine of preparation 3 (8.5 g, 28 mmol) was suspended in 2M aqueous sodium hydroxide solution (140 ml) and the mixture was heated at reflux for 9 hours. The reaction mixture was then cooled and acidified with concentrated hydrochloric acid (50 ml). The acidic mixture was extracted with ethyl acetate (250 ml), and the aqueous layer was then basified carefully with sodium carbonate (300 ml). The resulting precipitate was filtered off and dried over phosphorus pentoxide for 18 hours to yield the title compound (4.4 g, 67%) as a solid.

¹H NMR (400 MHz, CD₃OD): δ 1.93 (d, 2H), 2.53-2.64 (m, 2H), 3.03 (t, 2H), 3.42 (d, 2H), 4.48 (m, 1H), 6.81-6.86 (m, 2H), 7.31 (m, 1H); LRMS APCI⁺ m/z 236 [MH]⁺.

### Preparation 5

### tert-Butyl 4-(6-chloro[1,2,4]triazolo[4,3-b]pyridazin-3-yl)piperidine-1-carboxylate

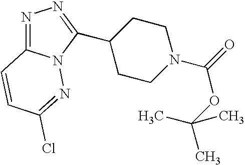

3,6-Dichloropyridazine (16.3 g, 67 mmol) and tert-butyl 4-(hydrazinocarbonyl)-1-piperidinecarboxylate (WO 00/39125, preparation 27, 10 g, 67 mmol) were suspended in isopropanol (150 ml) and the reaction mixture was heated at reflux for 48 hours. The solvent was evaporated and the residue was taken up in dichloromethane (50 ml). Di-tert-butyl dicarbonate (5 g, 23 mmol) and N-methyl morpholine (10 ml, 91 mmol) were added and the reaction mixture was stirred at room temperature for 15 minutes. The reaction mixture was then partitioned between 10% aqueous citric acid solution (50 ml) and ethyl acetate (500 ml). The aqueous layer was extracted three more times with ethyl acetate (3×100 ml). The organic solutions were combined, washed with brine (200 ml), dried over magnesium sulfate, filtered and concentrated under reduced pressure. The crude product was purified by column chromatography on silica gel using dichloromethane/methanol/aqueous ammonia as eluant (95:5:0.5 v/v/v) to yield the title compound (13.6 g, 60%)

¹H NMR (400 MHz, CDCl₃): δ 1.47 (s, 9H), 2.00-2.10 (m, 4H), 2.97-3.03 (m, 2H), 3.48 (m, 1H), 4.18-4.26 (m, 2H), 7.10 (d, 1H), 8.08 (d, 1H); LRMS ESI⁺ m/z 360 [MNa]⁺.

### Preparation 6

### tert-Butyl 4-([1,2,4]triazolo[4,3-b]pyridazin-3-yl)piperidine-1-carboxylate

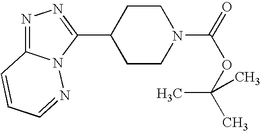

The piperidine of preparation 5 (6 g, 17.8 mmol) was dissolved in ethanol (200 ml) and aqueous ammonia (5 ml). The reaction mixture was then hydrogenated at 15 psi, at room temperature, for 1 hour over 5% Pd/C (1 g). The reaction mixture was then filtered through Arbocel®. The filtrate was concentrated under reduced pressure to give a brown solid. This was triturated with diethyl ether to yield the title compound (5.28 g, 98%).

¹H NMR (400 MHz, CDCl₃): δ 1.46 (s, 9H), 1.96-2.12 (m, 4H), 2.95-3.04 (m, 2H), 3.51 (m, 1H), 4.14-4.28 (m, 2H), 7.08 (dd, 1H), 8.10 (d, 1H), 8.34 (m, 1H); LRMS ESI⁺ m/z 304 [MH]⁺.

### Preparation 7

### 3-Piperidin-4-yl[1,2,4]triazolo[4,3-b]pyridazine dihydrochloride

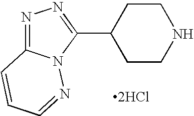

The piperidine of preparation 6 (5 g, 16.5 mmol) was suspended in dichloromethane (50 ml) and then 4M hydrochloric acid in dioxane (20 ml) was added. The reaction mixture was stirred at room temperature for 2 hours. It was determined that the reaction was not complete (by tlc: dichloromethane/methanol/aqueous ammonia as eluant (90:10:1 v/v/v)), so the reaction mixture was diluted with more dichloromethane (250 ml) and saturated with hydrogen chloride gas. The reaction mixture was then stirred at room temperature for a further 15 minutes after which time it was concentrated under reduced pressure. The resulting solid was azeotroped with 20% methanol in dichloromethane (3×200 ml), suspended in isopropanol and then filtered. The solid was triturated with diethyl ether and dried under reduced pressure to yield the title compound (4.4 g, 97%) as a white solid.

¹H NMR (400 MHz, CD₃OD): δ 2.26-2.36 (m, 2H), 2.49-2.53 (m, 2H), 3.32-3.36 (m, 2H), 3.56-3.61 (m, 2H), 3.98 (m, 1H), 7.98 (dd, 1H), 8.62 (d, 1H), 9.04 (m, 1H); LRMS ESI⁺ m/z 204 [MH]⁺.

### Preparation 8

### tert-Butyl 4-[(3-hydroxypyridin-2-yl)amino]piperidine-1-carboxylate

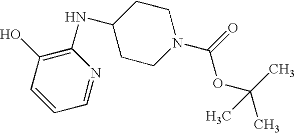

1-tert-Butyloxycarbonyl-4-piperidone (12.75 g, 64 mmol) was added to a solution of 2-amino-3-hydroxypyridine (4.7 g, 42 mmol) in dichloromethane (150 ml) and acetic acid (60 ml). Sodium sulfate (10 g, 70 mmol) was added and the reaction mixture was stirred at room temperature for 4 hours. Sodium triacetoxyborohydride (9.9 g, 47 mmol) was then added in 3 portions. The reaction mixture was then stirred at room temperature for 18 hours. The reaction was quenched carefully with saturated sodium bicarbonate solution (150 ml) and extracted with dichloromethane (500 ml). The organic layer was washed twice with saturated sodium bicarbonate solution (100 ml). The aqueous solutions were combined and extracted with ethyl acetate (2×150 ml). The organic layers were combined, dried over magnesium sulfate, filtered and concentrated under reduced pressure. The crude product was purified by column chromatography on silica gel using dichloromethane/methanol as eluant (97:3 to 93:7 v/v) to yield the title compound (1.24 g, 10%) as a solid.

¹H NMR (400 MHz, CDCl₃): δ 1.35-1.42 (m, 2H), 1.46 (s, 9H), 2.03-2.08 (m, 2H), 2.92-3.01 (m, 2H), 4.00-4.18 (m, 3H), 6.48 (m, 1H), 6.91 (d, 1H), 7.51 (m, 1H); LRMS APCI⁺ m/z 294 [MH]⁺.

### Preparation 9

### tert-Butyl 4-(2-oxo[1,3]oxazolo[4,5-b]pyridin-3(2H)-yl)piperidine-1-carboxylate

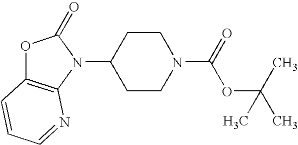

N,N′-Carbonyldiimidazole (343 mg, 2.12 mmol) was added to a solution of the piperidine of preparation 8 (600 mg, 1.93 mmol) in dichloromethane (6 ml). The reaction mixture was stirred at room temperature for 18 hours. The reaction mixture was then diluted with dichloromethane (50 ml) and washed with 1M hydrochloric acid (50 ml). The aqueous layer was extracted with dichloromethane (2×30 ml). The organics solutions were combined, washed with brine, dried over magnesium sulfate, filtered and concentrated under reduced pressure. The crude product was purified by column chromatography on silica gel using dichloromethane/acetone as eluant (98:2 to 92.5:7.5 v/v) to yield the title compound (555 mg, 90%) as a solid.

¹H NMR (400 MHz, CDCl₃): δ 1.48 (s, 9H), 1.82 (d, 2H), 2.50-2.60 (m, 2H), 2.76-2.88 (m, 2H), 4.22-4.45 (m, 3H), 7.04 (m, 1H), 7.39 (d, 1H), 8.09 (d, 1H); LRMS APCI⁺ m/z 220 [(M-BOC)H]⁺.

### Preparation 10

### 3-Piperidin-4-yl-[1,3]oxazolo[4,5-b]pyridin-2(3H)-one

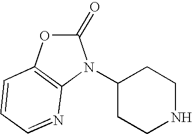

The piperidine of preparation 9 (550 mg, 1.72 mmol) was stirred at room temperature for 18 hours in dichloromethane (3 ml) and trifluoroacetic acid (3 ml). The reaction mixture was then concentrated under reduced pressure and the residue was partitioned between saturated sodium bicarbonate solution (25 ml) and 10% methanolic dichloromethane (50 ml). The layers were separated, and the aqueous layer was further extracted with 10% methanolic dichloromethane (50 ml). The organic layers were combined, washed with brine (2×20 ml), dried over magnesium sulfate, filtered and concentrated under reduced pressure to yield the title compound (300 mg, 80%) as a solid.

¹H NMR (400 MHz, CDCl₃): δ 1.86 (d, 2H), 2.32 (brs, 1H), 2.49-2.59 (m, 2H), 2.78 (t, 2H), 3.29 (d, 2H), 4.41 (m, 1H), 7.04 (t, 1H), 7.38 (d, 1H), 8.09 (d, 1H); LRMS APCI⁺ m/z 220 [MH]⁺.

### Preparation 11

### tert-Butyl 4-{[3-(2-ethoxy-2-oxoethoxy)pyridin-2-yl]amino}piperidine-1-carboxylate

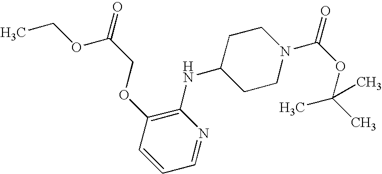

Sodium hydride (80 mg, 60% in mineral oil, 2.0 mmol) was added at 0° C. to a solution of the piperidine of preparation 8 (530 mg, 1.81 mmol) in tetrahydrofuran (9 ml). The reaction mixture was stirred at 0° C. for 30 minutes, and then ethyl bromoacetate (303 mg, 2.0 mmol) was added. The reaction mixture was stirred at room temperature for 18 hours, after which time, saturated sodium bicarbonate solution (50 ml) was added. The mixture was partitioned between ethyl acetate (200 ml) and saturated sodium bicarbonate solution (75 ml). The layers were separated and the aqueous phase was further extracted with ethyl acetate (150 ml). The combined organic layers were washed with brine (100 ml), dried over magnesium sulfate, filtered and concentrated under reduced pressure to yield the title compound (748 mg, 100%) as a solid.

¹H NMR (400 MHz, CDCl₃): δ 1.30 (t, 3H), 1.39-1.46 (m, 11H), 2.05-2.09 (m, 2H), 2.94-3.02 (m, 2H), 3.99-4.15 (m, 2H), 4.27 (q, 2H), 4.60 (s, 2H), 5.07 (m, 1H), 6.49 (t, 1H), 6.77 (d, 1H), 7.74 (m, 1H); LRMS APCI⁺ m/z 380 [MH]⁺.

### Preparation 12

### tert-Butyl 4-(3-oxo-2,3-dihydro-4H-pyrido[3,2-b][1,4]oxazin-4-yl)piperidine-1-carboxylate

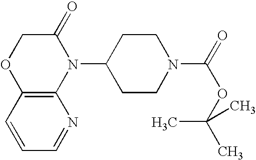

Lithium hydroxide monohydrate (85 mg, 2.05 mmol) in water (2 ml) was added to a solution of the compound from preparation 11 (748 mg, 1.97 mmol) in tetrahydrofuran (10 ml). The reaction mixture was stirred at room temperature for 18 hours, and then concentrated under reduced pressure. The residue was dissolved in N,N-dimethylformamide (7 ml) and O-(1H-benzotriazol-1-yl)-N,N,N′,N′-tetramethyluronium hexafluorophosphate (1.4 g, 3.7 mmol) was added. The reaction mixture was then stirred at room temperature for a further 15 hours, after which time it was partitioned between sodium bicarbonate solution (75 ml) and ethyl acetate (100 ml). The organic layer was washed with water (2×50 ml) and brine (50 ml). The aqueous layers were extracted again with ethyl acetate (50 ml). The combined organic layers were dried over magnesium sulfate, filtered and concentrated under reduced pressure. The crude product was purified by column chromatography on silica gel using dichloromethane/methanol/ammonia as eluant (98:2:0.2 v/v/v) to yield the title compound (446 mg, 72%) as a solid.

¹H NMR (400 MHz, CDCl₃): δ 1.48 (s, 9H), 1.63-1.67 (m, 2H), 2.67-2.86 (m, 4H), 4.16-4.32 (m, 2H), 4.58 (s, 2H), 5.04 (m, 1H), 6.93 (t, 1H), 7.22 (d, 1H), 7.99 (d, 1H); LRMS APCI⁺ m/z 234 [(M−BOC)H]⁺.

### Preparation 13

### 4-Piperidin-4-yl-2H-pyrido[3,2-b][1,4]oxazin-3(4H)-one

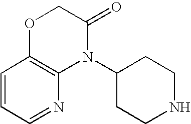

The title compound (300 mg, 97%) was prepared by a method similar to that described for preparation 10 using the piperidine of preparation 12.

¹H NMR (400 MHz, CDCl₃): δ 1.68-1.71 (m, 2H), 1.98 (s, 1H), 2.66-2.78 (m, 4H), 3.20-3.24 (m, 2H), 4.58 (s, 2H), 5.02 (m, 1H), 6.94 (m, 1H), 7.22 (d, 1H), 8.01 (m, 1H); LRMS APCI⁺ m/z 234 [MH]⁺.

### Preparation 14

### 1-(E/Z)-N′-Hydroxy-2-methylpropanimidamide

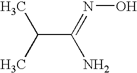

A mixture of hydroxylamine hydrochloride (60.33 g, 868 mmol) and triethylamine (121 ml, 868 mmol) was heated in methanol (300 ml) until homogeneous. A solution of isobutyronitrile (20 g, 289 mmol) in methanol (100 ml) was added dropwise over 30 minutes. The reaction mixture was then heated at reflux for 18 hours. The cooled reaction mixture was concentrated under reduced pressure to give a solid. This was taken up in 1N sodium hydroxide solution (300 ml) and extracted with ethyl acetate (3×300 ml). The aqueous layer was concentrated and further extracted with dichloromethane (600 ml). The combined organic extracts were dried over magnesium sulfate, filtered and concentrated under reduced pressure. The crude product was purified by column chromatography on silica gel using ethyl acetate as eluant to yield the title compound (20.4 g, 69%) as an oil.

¹H NMR (400 MHz, DMSO-D₆): δ 1.02 (d, 6H), 2.23 (m, 1H), 5.20 (brs, 2H), 8.65 (s, 1H); LRMS TS⁺ m/z 103 [MH]⁺.

### Preparation 15

### tert-Butyl 4-({[(1 E/Z)-N-hydroxy-2-methylpropanimidoyl]amino}carbonyl)piperidine-1-carboxylate

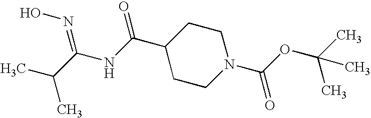

N,N′-Carbonyldiimidazole (24.75 g, 153 mmol) was added portionwise at room temperature to a solution of tert-butoxycarbonyl-isonipecotic acid (35.0 g, 153 mmol) in dichloromethane (400 ml). The reaction mixture was stirred at room temperature for 1 hour. A solution of the amidoxime of preparation 14 (20.0 g, 200 mmol) in dichloromethane (100 ml) was then added dropwise, after which time the reaction mixture was stirred at room temperature for 18 hours. The reaction mixture was quenched with water (300 ml) and the layers were separated. The organic layer was washed with 1M citric acid (800 ml) and saturated sodium bicarbonate solution (300 ml). The organic layer was dried over magnesium sulfate, filtered and decolourising charcoal was added to the filtrate. The mixture was stirred for 5 minutes, filtered and concentrated under reduced pressure to yield the title compound (35.05 g, 73%) as a solid.

¹H NMR (400 MHz, CDCl₃): δ 1.21 (d, 6H), 1.45 (s, 9H), 1.67-1.77 (m, 2H), 1.88-1.94 (m, 2H), 2.55-2.66 (m, 2H), 2.80-2.86 (m, 2H), 4.03-4.10 (m, 2H), 4.60 (brs, 1H); LRMS ESI⁺ m/z 336 [MNa]⁺.

### Preparation 16

### tert-Butyl 4-(3-isopropyl-[1,2,4]-oxadiazol-5-yl)piperidine-1-carboxylate

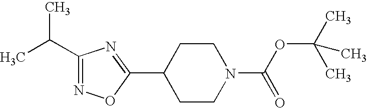

The piperidine of preparation 15 (35.0 g, 112 mmol) was heated at reflux in dioxane (300 ml) for 18 hours. The cooled mixture was then concentrated under reduced pressure to give an oil. This was azeotroped 3 times with ethyl acetate and dried under vacuum to yield the title compound (33 g, 100%) as an oil.

¹H NMR (400 MHz, CDCl₃): δ 1.33 (d, 6H), 1.46 (s, 9H), 1.76-1.86 (m, 2H), 2.01-2.06 (m, 2H), 2.90-2.98 (m, 2H), 3.03-3.10 (m, 2H), 4.06-4.11 (m, 2H); LRMS ESI⁺ m/z 318 [MNa]⁺.

### Preparation 17

### 4-(3-Isopropyl-[1,2,4]-oxadiazol-5-yl)piperidine hydrochloride

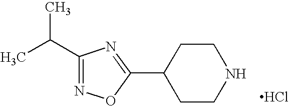

Hydrogen chloride gas was bubbled through a solution of the piperidine of preparation 16 (33.0 g, 112 mmol) in dichloromethane (50 ml) for 15 minutes. The reaction mixture was then stirred at room temperature for 3 hours. The mixture was concentrated under reduced pressure to yield the title compound as a white solid.

¹H NMR (400 MHz, CD₃OD): δ 1.31 (d, 6H), 2.01-2.12 (m, 2H), 2.32-2.37 (m, 2H), 3.06 (m, 1H), 3.16-3.23 (m, 2H), 3.41 (m, 1H), 3.44-3.50 (m, 2H); LRMS ESI⁺ m/z 196 [MH]⁺.

### Preparation 18

### tert-Butyl 4-(5-isopropyl-1,2,4-oxadiazol-3-yl)piperidine-1-carboxylate

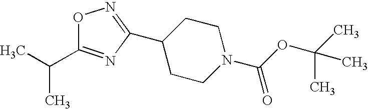

tert-Butyl 4-[(E/Z)-amino(hydroxyimino)methyl]piperidine-1-carboxylate (described in WO2000/039125, prep 28, 24.3 g, 100 mmol), triethylamine (15.3 ml, 110 mmol) and 4-dimethylaminopyridine (1 g, 8 mmol) were mixed in dichloromethane (500 ml) and cooled to 5° C. Isobutyryl chloride (11.5 ml, 110 mmol) was then added dropwise over 15 minutes, keeping the internal temperature at about 10° C. The reaction mixture was warmed to room temperature and then stirred for 18 hours. It was then diluted with dichloromethane (200 ml) and the organic layer was washed with 10% aqueous citric acid solution (2×300 ml) and saturated sodium bicarbonate solution (2×250 ml). The organic layer was then washed with brine (150 ml), dried over magnesium sulfate, filtered and concentrated under reduced pressure to yield the crude ring-opened intermediate. This was taken up in toluene (500 ml) and the mixture was heated at reflux under Dean-Stark conditions for 18 hours. The reaction mixture was then allowed to cool and it was concentrated under reduced pressure. The crude product was purified by column chromatography on silica gel using dichloromethane:ethyl acetate as eluant (100:0 to 80:20 v/v) to yield the title compound (29.5 g, 100%) as an oil.

¹H NMR (400 MHz, CDCl₃): δ 1.33 (d, 6H), 1.40 (s, 9H), 1.65-1.75 (m, 2H), 1.90-1.95 (m, 2H), 2.82-2.90 (m, 3H), 3.13 (m, 1H), 4.03-4.09 (m, 2H); LRMS ESI⁺ m/z 296 [MH]⁺.

### Preparation 19

### 4-(5-Isopropyl-1,2,4-oxadiazol-3-yl)piperidine hydrochloride

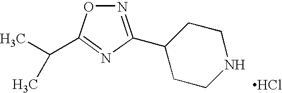

Hydrogen chloride gas was bubbled through a cooled solution of the piperidine of preparation 18 (29.5 g, 100 mmol) in ethyl acetate (500 ml) for 10 minutes. The reaction mixture was then stirred at room temperature for a further 3 hours. The reaction mixture was then concentrated under reduced pressure to give a solid, which was suspended in ethyl acetate (150 ml) and stirred for 5 minutes. The solid was filtered and dried under high vacuum to yield the title compound (21.4 g, 92%) as an off-white solid (mp 140-144° C.).

¹H NMR (400 MHz, CD₃OD): δ 1.39 (d, 6H), 1.97-2.11 (m, 2H), 2.25-2.33 (m, 2H), 3.15-3.29 (m, 4H), 3.44-3.50 (m, 2H); LRMS ESI⁺ m/z 196 [MH]⁺.

### Preparations 20 to 30

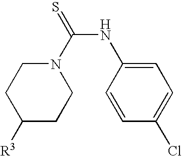

The appropriate piperidine or piperidine hydrochloride (1 eq.) was added to a solution of 4-chlorophenyl isothiocyanate (1 eq.) in ethanol (8.0-20.5 mlmmol⁻¹). {when the hydrochloride salt of the piperidine was used, 1-3 eq. triethylamine were also added}. The reaction mixture was stirred at room temperature for 2 hours. The mixture was then concentrated under reduced pressure and the residue was triturated with ethyl acetate and filtered off to yield the title compound as an off-white solid.

| Prep no | R³ | Data |
|---|---|---|
| 20^{A} | 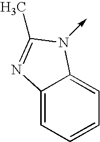 | ¹H NMR (400 MHz, CD₃OD): δ 1.97-2.01 (m, 2H), 2.51-2.60 (m,2H), 2.65 (s, 3H), 3.31-3.35 (m, 2H), 4.75 (m, 1H), 4.98 (d,2H), 7.16-7.23 (m, 2H), 7.31 (s, 4H), 7.53 (d, 1H), 7.57 (d, 1H);LRMS APCI⁺ m/z 385 [MH]⁺. |
| 21^{B} | 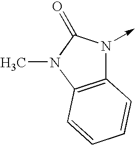 | ¹H NMR (400 MHz, DMSO-D₆): δ 1.78 (d, 2H), 2.30-2.39 (m,2H), 3.21 (t, 2H), 3.30 (s, 3H), 4.56 (m, 1H), 4.90 (d, 2H), 7.04-7.07 (m, 2H), 7.13 (m, 1H), 7.25 (m, 1H), 7.32-7.37 (m, 4H),9.51 (s, 1H); LRMS APCI⁺ m/z 401 [MH]⁺. |
| 22 | 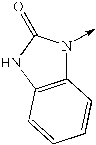 | ¹H NMR (400 MHz, DMSO-D₆): δ 1.78 (d, 2H), 2.30-2.38 (m,2H), 3.20 (t, 2H), 4.50 (m, 1H), 4.89 (d, 2H), 6.96-7.01 (m, 3H),7.18 (m, 1H), 7.33-7.36 (m, 4H), 9.44 (s, 1H), 10.87 (s, 1H);LRMS APCI⁺ m/z 387 [MH]⁺. |
| 23 | 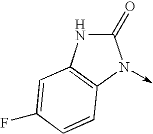 | LRMS APCI⁺ m/z 405 [MH]⁺. |
| 24 | 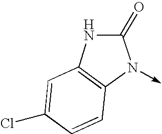 | ¹H NMR (400 MHz, DMSO-D₆): δ 1.78 (d, 2H), 2.25-2.34 (m,2H), 3.18 (t, 2H), 4.49 (m, 1H), 4.89 (d, 2H), 6.99 (s, 1H), 7.04(d, 1H), 7.21 (d, 1H), 7.34 (s, 4H); LRMS APCI⁺ m/z 421 [M]⁺. |
| 25^{C} | 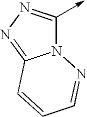 | ¹H NMR (400 MHz, CDCl₃): δ 2.23-2.27 (m, 4H), 3.48-3.55 (m,2H), 3.74 (m, 1H), 4.60-4.65 (m, 2H), 7.10-7.16 (m, 3H), 7.29(d, 2H), 7.53 (s, 1H), 8.08 (d, 1H), 8.37 (m, 1H); LRMS ESI⁺m/z 395 [MNa]⁺. |
| 26 | 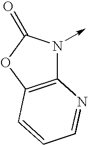 | ¹H NMR (400 MHz, CDCl₃): δ 1.91-1.96 (m, 2H), 2.67-2.78 (m,2H), 3.17 (t, 2H), 4.56 (m, 1H), 4.81 (d, 2H), 7.07 (m, 1H), 7.16(d, 2H), 7.24 (s, 1H), 7.32 (d, 2H), 7.41 (d, 1H), 8.10 (d, 1H);LRMS APCI⁺ m/z 389 [MH]⁺. |
| 27 | 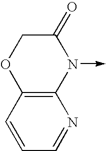 | ¹H NMR (400 MHz, CDCl₃): δ 1.77-7.81 (m, 2H), 2.82-2.93 (m,2H), 3.16-3.24 (m, 2H), 4.60 (s, 2H), 4.72 (d, 2H), 5.20 (m,1H), 6.98 (m, 1H), 7.12 (s, 1H), 7.16 (d, 2H), 7.25 (m, 1H),7.31 (d, 2H), 8.03 (d, 1H); LCMS APCI⁺ m/z 403 [MH]⁺. |
| 28^{D} | 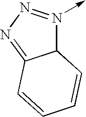 | ¹H NMR (400 MHz, CDCl₃): δ 2.29-2.35 (m, 2H), 2.48-2.57 (m,2H), 3.52-3.59 (m, 2H), 4.70 (d, 2H), 5.00 (m, 1H), 7.17 (d,2H), 7.33 (d, 2H), 7.41 (t, 1H), 7.50-7.58 (m, 3H), 8.06 (d, 1H);LCMS APCI⁺ m/z 372 [MH]⁺. |
| 29^{D} | 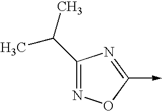 | ¹H NMR (400 MHz, CDCl₃): δ 1.33 (d, 6H), 1.97-2.07 (m, 2H),2.14-2.20 (m, 2H), 3.07 (m, 1H), 3.24 (m, 1H), 3.37-3.44 (m,2H), 4.42-4.48 (m, 2H), 7.10 (d, 2H), 7.30 (d, 2H); LCMSAPCI⁺ m/z 365 [MH]⁺. |
| 30 | 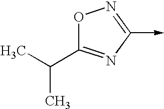 | ¹H NMR (400 MHz, CDCl₃): δ 1.39 (d, 6H), 1.93-2.02 (m, 2H),2.08-2.14 (m, 2H), 3.10 (m, 1H), 3.20 (m, 1H), 3.33-3.39 (m,2H), 4.47-4.52 (m, 2H), 7.08-7.13 (m, 3H), 7.30 (d, 2H);LCMS APCI⁺ m/z 365 [MH]⁺. |

A= 2-methyl-1-(4-piperidinyl)-1H-benzimidazole hydrochloride (described in J. Het. Chem (1983), 20(3), 565
B= 4-(2-keto-3-methyl-1-benzimidazolinyl)piperidine hydrochloride (described in patent WO9528397A1)
C= dichloromethane was used as the reaction solvent. The crude reaction mixture was partitioned between dichloromethane and 50% aqueous ammonia, the organic phase was dried over magnesium sulfate and evaporated under reduced pressure. The product was isolated after crystallisation from ethanol.
D= crude product was purified by column chromatography on silica gel using ethyl acetate:pentane as eluant.

### Preparations 31 to 40

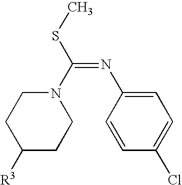

Potassium tert-butoxide (1 eq.) was added to a suspension of the appropriate thiourea (1 eq.) from preparations 20 to 24 and 26 to 30 in tetrahydrofuran (3.5 to 8.7 mlmmol⁻¹) and the solution was stirred for 15 minutes. Methyl tosylate (1 eq.) was then added and the reaction mixture was stirred at room temperature for a further 18 hours. The reaction mixture was then partitioned between water and ethyl acetate, the layers were separated and the aqueous phase was further extracted with ethyl acetate. The combined organic solutions were washed with brine, dried over magnesium sulfate, filtered and evaporated under reduced pressure to provide the title compounds.

| Prep no | R³ | Data |
|---|---|---|
| 31^{A} | 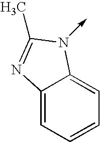 | ¹H NMR (400 MHz, CD₃OD): δ 1.95-2.01 (m, 2H), 2.18 (s, 3H),2.54-2.63 (m, 2H), 2.67 (s, 3H), 3.15-3.22 (m, 2H), 4.46-4.50(m, 2H), 4.67 (m, 1H), 6.88 (d, 2H), 7.21-7.27 (m, 4H), 7.56 (d,1H), 7.61 (d, 1H); LRMS APCI⁺ m/z 399 [MH]⁺. 81% yield |
| 32 | 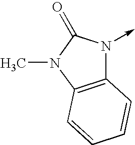 | ¹H NMR (400 MHz, CDCl₃): δ 1.89-1.93 (m, 2H), 2.13 (s, 3H),2.49-2.56 (m, 2H), 3.10-3.19 (m, 2H), 3.42 (s, 3H), 4.50-4.58(m, 3H), 6.94-7.01 (m, 2H), 7.08-7.19 (m, 3H), 7.24 (m, 1H),7.35 (m, 1H), 7.79 (m, 1H); LRMS APCI⁺ m/z 415 [MH]⁺.53% yield |
| 33^{A} | 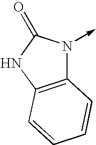 | ¹H NMR (400 MHz, CD₃OD): δ 1.84-1.87 (m, 2H), 2.16 (s, 3H),2.50-2.59 (m, 2H), 3.11 (t, 2H), 4.44-4.54 (m, 3H), 6.86 (d,2H), 7.07-7.10 (m, 3H), 7.22-7.27 (m, 3H); LRMS APCI⁺ m/z401 [MH]⁺. 62% yield |
| 34 | 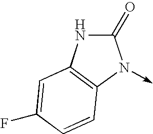 | ¹H NMR (400 MHz, CDCl₃): δ 1.93 (d, 2H), 2.11 (s, 3H), 2.45-2.49 (m, 2H), 3.09 (t, 2H), 4.49-4.55 (m, 3H), 6.80 (m, 1H),6.87-6.91 (m, 2H), 7.06 (m, 1H), 7.24 (m, 1H), 7.35 (d, 1H),7.79 (d, 1H), 9.63 (s, 1H); LRMS APCI⁺ m/z 419 [MH]⁺.73% yield |
| 35 | 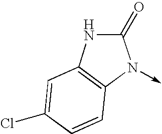 | ¹H NMR (400 MHz, CDCl₃): δ 1.89-1.94 (m, 2H), 2.12 (s, 3H),2.44-2.51 (m, 2H), 3.04-3.16 (m, 2H), 4.50-4.56 (m, 3H), 6.93(m, 1H), 7.05-7.07 (m, 2H), 7.11 (m, 1H), 7.25-7.27 (m, 2H),7.32 (m, 1H), 9.27 (s, 1H); LRMS APCI⁺ m/z 435 [M]⁺.100% yield |
| 36 | 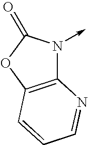 | ¹H NMR (400 MHz, CDCl₃): δ 1.92 (d, 2H), 2.09 (s, 3H), 2.63-2.72 (m, 2H), 3.01 (t, 2H), 4.48-4.54 (m, 3H), 6.85 (d, 2H),7.07 (t, 1H), 7.22 (d, 2H), 7.79 (d, 1H), 8.11 (d, 1H); LRMSAPCI⁺ m/z 403 [MH]⁺. 100% yield |
| 37 | 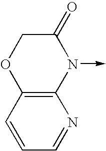 | ¹H NMR (400 MHz, CDCl₃): δ 1.73-1.78 (m, 2H), 2.10 (s, 3H),2.80-2.90 (m, 2H), 2.99 (t, 2H), 4.46 (d, 2H), 4.59 (s, 2H), 5.14(m, 1H), 6.85 (d, 2H), 6.96 (m, 1H), 7.20-7.23 (m, 3H), 8.02 (d,1H); LRMS APCI⁺ m/z 417 [MH]⁺. 100% yield |
| 38 | 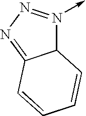 | ¹H NMR (400 MHz, CDCl₃): δ 2.12 (s, 3H), 2.26-2.31 (m, 2H),2.45-2.55 (m, 2H), 3.21-3.28 (m, 2H), 4.53 (d, 2H), 4.92 (m,1H), 6.88 (d, 2H), 7.24 (m, 2H), 7.40 (t, 1H), 7.51 (t, 1H), 7.59(d, 1H), 8.09 (d, 1H); LRMS ESI⁺ m/z 386 [MH]⁺ . 100% yield |
| 39 | 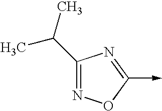 | ¹H NMR (400 MHz, CDCl₃): δ 1.33 (d, 6H), 1.90-2.00 (m, 2H),2.07 (s, 3H), 2.12-2.18 (m, 2H), 3.04-3.20 (m, 4H), 4.26-4.31(m, 2H), 6.82 (d, 2H), 7.21 (d, 2H); LRMS APCI⁺ m/z 379[MH]⁺. 95% yield |
| 40 | 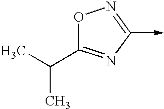 | ¹H NMR (400 MHz, CDCl₃): δ 1.40 (d, 6H), 1.85-1.95 (m, 2H),2.06-2.13 (m, 5H), 2.99-3.13 (m, 3H), 3.21 (m, 1H), 4.27-4.33(m, 2H), 6.82 (d, 2H), 7.21 (d, 2H); LRMS APCI⁺ m/z 379[MH]⁺. 96% yield |

A=The mixture was poured onto water and a white precipitate was formed. This was filtered off, then triturated with diethyl ether to yield the title compound

### Preparation 41

### 4-(1,2-Benzisothiazol-3-yl)-N-(4-chlorophenyl)piperazine-1-carbothioamide

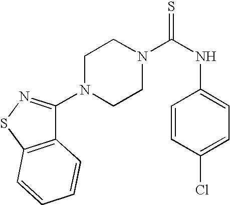

The title compound was prepared by a method similar to that described for preparations 20 to 30, using 4-chlorophenyl isothiocyanate and 3-piperazin-1-yl-1,2-benzisothiazole (described in J. Med. Chem. (1986), 29(3), 359-369).

¹H NMR (400 MHz, CDCl₃): δ 3.66-3.69 (m, 4H), 4.07-4.10 (m, 4H), 7.16 (d, 2H), 7.31 (d, 2H), 7.38 (t, 1H), 7.49 (t, 1H), 7.83 (d, 1H), 7.89 (d, 1H); LRMS APCI⁺ m/z 389 [MH]⁺.

### Preparation 42

### tert-Butyl (1-{[(4-chlorophenyl)amino]carbonothioyl}piperidin-4-yl)carbamate

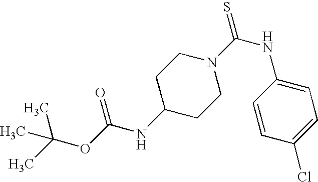

The title compound (42.3 g, 91%) was prepared by a method similar to that described for preparations 20 to 30, using 4-chlorophenyl isothiocyanate and 4-N-butoxycarbonyl-aminopiperidine.

¹H NMR (400 MHz, CDCl₃): δ 1.29-1.54 (m, 11H), 2.01 (m, 2H), 3.20 (m, 2H), 3.74 (br s, 1H), 4.35-4.55 (m, 3H), 7.09 (d, 2H), 7.17 (br s, 1H), 7.32 (d, 2H).

### Preparation 43

### Methyl 4-(1,2-benzisothiazol-3-yl)-N-(4-chlorophenyl)piperazine-1-carbimidothioate

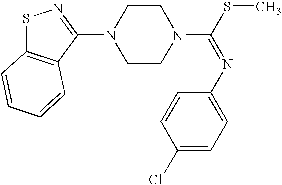

The title compound (100% yield) was prepared by a method similar to that described for preparations 31 to 40, using the thioamide of preparation 41.

¹H NMR (400 MHz, CDCl₃): δ 2.09 (s, 3H), 3.57-3.60 (m, 4H), 3.82-3.86 (m, 4H), 6.85 (d, 2H), 7.23 (d, 2H), 7.38 (t, 1H), 7.49 (t, 1H), 7.83 (d, 1H), 7.91 (d, 1H); LRMS APCI⁺ m/z 403 [MH]⁺.

### Preparation 44

### Methyl 4-[(tert-butoxycarbonyl)amino]-N-(4-chlorophenyl)piperidine-1-carbimidothioate

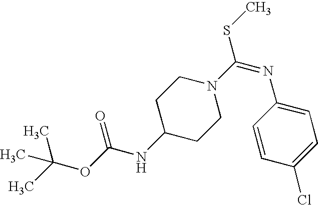

The title compound (43.6 g, 100%) was prepared by a method similar to that described for preparations 31 to 40, using the thioamide of preparation 42.

¹H NMR (400 MHz, CDCl₃): δ 1.34-1.52 (m, 11H), 2.00 (m, 2H), 2.05 (s, 3H), 3.04 (m, 2H), 3.68 (br s, 1H), 4.19 (m, 2H), 4.50 (br s, 1H), 6.80 (d, 2H), 7.20 (d, 2H); LRMS ESI⁺ m/z 384 [MH]⁺.

### Preparation 45

### 3-[(3-endo)-8-Azabicyclo[3.2.1]oct-3-yl]-2-methyl-3H-imidazo[4,5-c]pyridine dihydrochloride

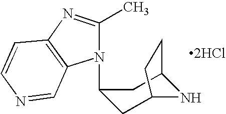

3-endo-(8-Acetyl-8-azabicyclo[3.2.1]oct-3-yl)-2-methyl-3H-imidazo[4,5-c]pyridine (described in WO 2003/084954 prep 8, 7.4 g, 26 mmol) was heated at reflux in 6N aqueous hydrochloric acid (100 ml) for 18 hours. The reaction mixture was then concentrated under reduced pressure and dried in vacuo to yield the title compound as a yellow solid, which was used without further purification.

¹H NMR (400 MHz, CD₃OD): δ 2.27-2.36 (m, 6H), 2.82-2.88 (m, 2H), 2.91 (s, 3H), 4.26-4.30 (m, 2H), 5.34 (m, 1H), 8.14 (d, 1H), 8.57 (d, 1H), 9.43 (s, 1H); LRMS APCI⁺ m/z 243 [MH]⁺.

### Preparation 46

### (3-endo)-N-(4-Chlorophenyl)-3-(2-methyl-3H-imidazo[4,5-c]pyridin-3-yl)-8-azabicyclo[3.2.1]octane-8-carbothioamide

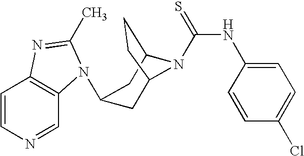

The title compound (2.5 g, 100%) was prepared by a method similar to that described for preparations 20-31 using 4-chlorophenyl isothiocyanate and the tropane of preparation 45.

¹H NMR (400 MHz, CD₃OD): δ 2.13-2.18 (m, 2H), 2.27 (t, 2H), 2.33-2.38 (m, 2H), 2.68 (s, 3H), 2.83-2.90 (m, 2H), 4.46 (m, 1H), 5.03-5.13 (m, 2H), 7.35 (d, 2H), 7.41 (d, 2H), 7.62 (d, 1H), 8.33 (d, 1H), 8.94 (s, 1H); LRMS APCI⁺ m/z 412 [MH]⁺.

### Preparation 47

### Methyl (3-endo)-N-(4-chlorophenyl)-3-(2-methyl-3H-imidazo[4,5-c]pyridin-3-yl)-8-azabicyclo[3.2.1]octane-8-carbimidothioate

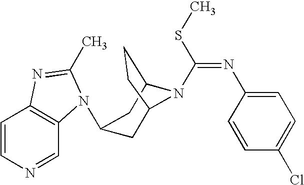

The title compound (2.5 g, 97%) was prepared by a method similar to that described for preparations 31 to 40 using the thiourea of preparation 46.

¹H NMR (400 MHz, CDCl₃): δ 94-1.99 (m, 2H), 2.14-2.21 (m, 5H), 2.29-2.34 (m, 2H), 2.65 (s, 3H), 2.68-2.76 (m, 2H), 4.58-4.67 (m, 3H), 6.87 (d, 2H), 7.25 (d, 2H), 7.61 (d, 1H), 8.40 (d, 1H), 8.87 (s, 1H); LRMS APCI⁺ m/z 426 [MH]⁺.

### Preparation 48

### tert-Butyl {1-[4-(4-chlorophenyl)-5-methyl-4H-1,2,4-triazol-3-yl]piperidin-4-yl}carbamate

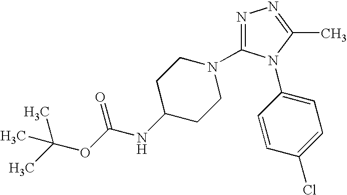

Acetic hydrazide (16.9 g, 228 mmol), followed by trifluoroacetic acid (4.4 ml, 57.1 mmol), was added to a solution of the compound of preparation 44 (43.6 g, 114 mmol) in tetrahydrofuran (250 ml) and the reaction mixture was heated under reflux for 7 hours. The cooled reaction mixture was then washed with dilute ammonia solution (100 ml), the layers were separated and the aqueous phase was extracted further with ethyl acetate (100 ml). The combined organic solutions were dried over magnesium sulfate, filtered and evaporated under reduced pressure. The residue was triturated with ether (100 ml) and the resulting crystals were filtered off and dried in vacuo to afford the title compound (32.4 g, 72%).

¹H NMR (400 MHz, CDCl₃): δ 1.32 (m, 2H), 1.40 (s, 9H), 1.85 (m, 2H), 2.22 (s, 3H), 2.84 (m, 2H), 3.24 (m, 2H), 3.52 (m, 1H), 4.44 (m, 1H), 7.24 (d, 2H), 7.51 (d, 2H); LRMS APCI⁺ m/z 392 [MH]⁺

### Preparation 49

### 1-[4-(4-Chlorophenyl)-5-methyl-4H-1,2,4-triazol-3-yl]piperidin-4-amine dihydrochloride

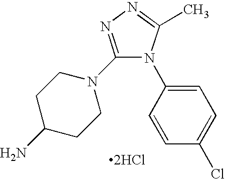

A suspension of the compound of preparation 48 (32.3 g, 82.5 mmol), in methanol (250 ml), and 4N hydrochloric acid, in dioxan (40 ml), was heated at 50° C. for 3 hours. The reaction mixture was then concentrated under reduced pressure and the residue was slurried in tetrahydrofuran (50 ml). The resulting solid was filtered off and dried in vacuo to provide the title compound (33.6 g, 100%).

¹H NMR (400 MHz, CD₃OD): δ 1.65 (m, 2H), 1.96 (m, 2H), 2.36 (s, 3H), 3.07 (m, 2H), 3.36 (m, 1H), 3.47 (m, 2H), 7.66 (d, 2H), 7.75 (d, 2H); LRMS APCI⁺ m/z 292 [MH]⁺

### Preparation 50

### N-{1-[4-(4-Chlorophenyl)-5-methyl-4H-1,2,4-triazol-3-yl]piperidin-4-yl}-3-nitropyridin-2-amine

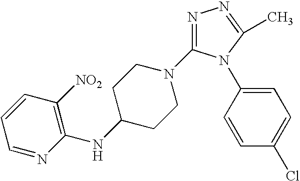

Triethylamine (0.84 ml, 6.3 mmol) was added to a suspension of the amine of preparation 49 (1 g, 2.01 mmol) in tetrahydrofuran (10 ml). 2-Chloro-3-nitropyridine (319 mg, 2.01 mmol) was added and the reaction mixture was stirred at room temperature for 18 hours under nitrogen. N,N-Dimethylformamide (3 drops) was added, for solubility, and the reaction mixture was heated at 65° C. for 24 hours. The reaction mixture was then concentrated under reduced pressure. The residue was partitioned between dichloromethane (50 ml) and water (50 ml). The organic solution was then washed with brine (50 ml), dried over magnesium sulfate, filtered and concentrated under reduced pressure. The crude product was purified by column chromatography on silica gel using dichloromethane/methanol/aqueous ammonia as eluant (95:5:0.5 v/v/v) to yield the title compound (200 mg, 24%)

¹H NMR (400 MHz, CDCl₃): δ 1.43-1.52 (m, 2H), 1.96-2.00 (m, 2H), 2.19 (s, 3H), 2.93 (t, 2H), 3.28 (d, 2H), 4.23 (m, 1H), 6.58 (dd, 1H), 7.23 (d, 2H), 7.48 (d, 2H), 8.05 (d, 1H), 8.30-8.35 (m, 2H); LRMS APCI⁺ m/z 414 [MH]⁺.

### Preparation 51

### N²-{1-[4-(4-Chlorophenyl)-5-methyl-4H-1,2,4-triazol-3-yl]piperidin-4-yl}pyridine-2,3-diamine

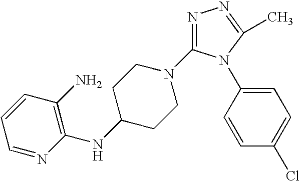

Raney® Nickel (20 mg) was added to a solution of the nitropyridine of preparation 50 (200 mg, 0.48 mmol) in ethanol (7 ml) and tetrahydrofuran (15 ml). The reaction mixture was then hydrogenated at 30 psi, at room temperature, for 16 hours. It was then filtered through glass fibre paper and the filtrate was concentrated under reduced pressure. The crude product was purified by column chromatography on silica gel using dichloromethane/methanol/aqueous ammonia as eluant (90:10:1 v/v/v) to yield the title compound (176 mg, 96%)

¹H NMR (400 MHz, DMSO-D₆): δ 1.29-1.38 (m, 2H), 1.79-1.83 (m, 2H), 2.11 (s, 3H), 2.74-2.79 (m, 2H), 3.13-3.17 (m, 2H), 3.89 (m, 1H), 6.28 (dd, 1H), 6.61 (d, 1H), 7.28 (d, 1H), 7.53 (d, 2H), 7.64 (d, 2H); LRMS APCI⁺ m/z 384 [MH]⁺.

### Preparation 52

### 1-[4-(4-Chlorophenyl)-5-methyl-1H-1,2,4-triazol-3-yl]-N-(2-nitrophenyl)piperidin-4-amine

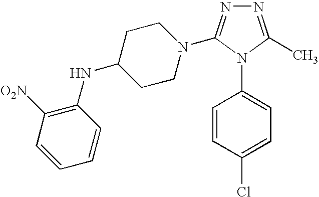

Triethylamine (1.58 ml, 11.3 mmol) was added to a suspension of the piperidine of preparation 49 (3 g, 10.3 mmol) in tetrahydrofuran (40 ml). 2-Fluoro-nitrobenzene (1.08 ml, 10.3 mmol) was added and the reaction mixture was heated at reflux for 23 hours, under nitrogen, and then allowed to cool. The resulting precipitate was filtered off, and the filtrate was concentrated under reduced pressure. The residue was partitioned between dichloromethane (50 ml) and water (50 ml). The organic layer was washed with brine (50 ml), dried over magnesium sulfate, filtered and concentrated under reduced pressure to give an orange solid. This was triturated with diethyl ether to yield the title compound (2.07 g, 49%)

¹H NMR (400 MHz, CDCl₃): δ 1.49-1.59 (m, 2H), 2.03-2.07 (m, 2H), 2.26 (s, 3H), 2.97 (t, 2H), 3.33-3.38 (m, 2H), 3.61 (m, 1H), 6.63 (t, 1H), 6.82 (d, 1H), 7.28 (d, 2H), 7.41 (t, 1H), 7.54 (d, 2H), 8.02 (d, 1H), 8.16 (d, 1H); LRMS ESI⁺ m/z 435 [MNa]⁺.

### Preparation 53

### N-{1-[4-(4-Chlorophenyl)-5-methyl-4H-1,2,4-triazol-3-yl]piperidin-4-yl}benzene-1,2-diamine

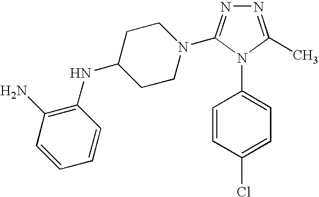

The title compound (479 mg, 100%) was prepared by a method similar to that described for preparation 51 using the piperidine of preparation 52.

¹H NMR (400 MHz, CDCl₃): δ 1.36-1.46 (m, 2H), 1.98-2.02 (m, 2H), 2.25 (s, 3H), 2.89-2.96 (m, 2H), 3.29-3.36 (m, 2H), 3.72 (m, 1H), 6.61-6.78 (m, 4H), 7.28 (d, 2H), 7.52 (d, 2H); LRMS ESI⁺ m/z 405 [MNa]⁺.

### Preparation 54

### 1-{1-[4-(4-Chlorophenyl)-5-methyl-4H-1,2,4-triazol-3-yl]piperidin-4-yl}-1,3-dihydro-2,1,3-benzothiadiazole-2,2-dioxide

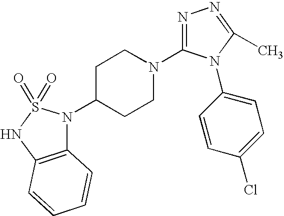

Sulfamide (233 mg, 2.41 mmol) was added to a solution of the amine of preparation 53 (465 mg, 1.21 mmol) in pyridine (3 ml). The reaction mixture was heated at reflux for 18 hours, after which time, it was concentrated under reduced pressure. The residue was partitioned between ethyl acetate (25 ml) and dilute hydrochloric acid (25 ml), and the layers were separated. The organic layer was dried over magnesium sulfate, filtered and concentrated under reduced pressure. The crude product was purified by column chromatography on silica gel using dichloromethane/methanol/aqueous ammonia as eluant (90:10:1 v/v/v) to yield the title compound (130 mg, 24%) after trituration with diethyl ether.

¹H NMR (400 MHz, CD₃OD): δ 1.95-1.99 (m, 2H), 2.15-2.23 (m, 5H), 2.96 (t, 2H), 3.42 (d, 2H), 4.06 (m, 1H), 6.80 (m, 1H), 6.85-6.89 (m, 2H), 6.94 (m, 1H), 7.52 (d, 2H), 7.65 (d, 2H); LRMS ESI⁺ m/z 445 [MH]⁺.

### Preparation 55

### 5-Fluoro-3-nitropyridin-2-ol

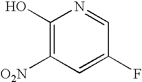

To a solution of 5-fluoro-2-hydroxypyridine (200 mg, 1.77 mmol) in concentrated sulfuric acid (900 μl) was added, dropwise over 15 minutes, a premixed solution of concentrated sulfuric acid (900 μl) and fuming nitric acid (170 μl). The internal temperature rose by up to 28° C. The reaction mixture was then heated at 65° C. for 2.5 hours. The cooled mixture was poured onto ice-water, and the pH of the mixture was adjusted to 2.5 with sodium carbonate. It was then extracted with ethyl acetate (2×25 ml). The aqueous layer was concentrated and extracted again with a mixture of tetrahydrofuran (25 ml) and ethyl acetate (25 ml). The organic layers were combined, dried over magnesium sulfate, filtered and concentrated under reduced pressure to yield the title compound (112 mg, 40%) as a solid.

¹H NMR (400 MHz, DMSO-D₆): δ 8.22 (dd, 1H), 8.60 (dd, 1H); LRMS APCI⁻ m/z 157 [M-H]⁻.

### Preparation 56

### 2-Chloro-5-Fluoro-3-nitropyridine

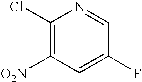

A mixture of the pyridine of preparation 55 (105 mg, 0.66 mmol), phosphorus oxychloride (1 ml) and N,N-dimethylformamide (10 μl, catalytic) was heated at 110° C. for 18 hours. The cooled reaction mixture was then concentrated under reduced pressure. The crude product was purified by column chromatography on silica gel using dichloromethane/acetonitrile as eluant (100:0 to 50:50 v/v) to yield the title compound (48 mg, 41%) as a solid.

¹H NMR (400 MHz, CDCl₃): δ 8.03 (dd, 1H), 8.55 (d, 1H).

### Preparation 57

### N-{1-[4-(4-Chlorophenyl)-5-methyl-4H-1,2,4-triazol-3-yl]piperidin-4-yl}-5-fluoro-3-nitropyridin-2-amine

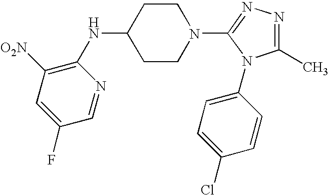

The amine of preparation 49 (166 mg, 0.464 mmol) was dissolved in dichloromethane (10 ml) and the solution was treated with 1N sodium hydroxide solution (10 ml). The layers were separated, and the organic phase was dried over magnesium sulfate, filtered and evaporated under reduced pressure. The pyridine of preparation 56 (41 mg, 0.23 mmol) was added to a solution of this amine in tetrahydrofuran (3 ml). The reaction mixture was heated at reflux for 18 hours. The cooled mixture was then concentrated under reduced pressure. The crude product was purified by column chromatography on silica gel using dichloromethane/methanol/ammonia as eluant (99:1:0 to 95:5:0.5 v/v/v) to yield the title compound (80 mg, 80%) as a solid.

¹H NMR (400 MHz, CD₃OD): δ 1.52-1.62 (m, 2H), 1.96-2.02 (m, 2H), 2.23 (s, 3H), 2.95 (t, 2H), 3.29-3.33 (m, 2H), 4.28 (m, 1H), 7.51 (d, 2H), 7.64 (d, 2H), 8.29 (dd, 1H), 8.42 (s, 1H); LRMS APCI⁺ m/z 432 [MH]⁺.

### Preparation 58

### N²-{1-[4-(4-Chlorophenyl)-5-methyl-4H-1,2,4-triazol-3-yl]piperidin-4-yl}-5-fluoropyridine-2,3-diamine

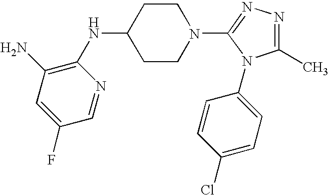

The title compound (90 mg, 100%, crude) was prepared by a method similar to that described for preparation 51 using the compound of preparation 57.

LRMS APCI⁺ m/z 402 [MH]⁺.

### Preparation 59

### 4-(5-Methyl-[1,3,4]oxadiazol-2-yl)-piperidine-1-carboxylic acid tert-butyl ester

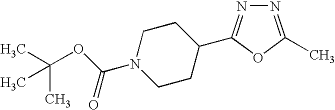

To a solution of tert-butyl 4-(hydrazinocarbonyl)-1-piperidinecarboxylate (see WO 00/39125, preparation 27) (9.0 g, 37 mmol), in tetrahydrofuran (40 ml), was added dimethylformamide dimethyl acetal (8.1 ml, 55.4 mmol). The reaction mixture was stirred at 50° C. for 4 hours, under nitrogen. The solvent was then removed under reduced pressure, the residue was dissolved in toluene (40 ml), and para-toluenesulfonic acid (400 mg, 2.1 mmol) was added. The reaction mixture was heated at 100° C., under nitrogen, for 18 hours, after which time the cooled reaction mixture was concentrated under reduced pressure and the residue was partitioned between dichloromethane (200 ml) and an aqueous solution of sodium bicarbonate (150 ml). The organic phase was separated and dried over magnesium sulfate, filtered and concentrated under reduced pressure. The residue was purified by column chromatography on silica gel using dichloromethane/methanol as eluant (98:2 v/v to 95:5 v/v) to yield the title compound (8.07 g, 81%) as a white solid.

¹H NMR (400 MHz, CD₃OD): δ 1.42 (s, 9H), 1.70 (m, 2H), 2.05 (m, 2H), 2.50 (s, 3H), 3.00 (m, 2H), 3.15 (m, 1H), 4.05 (m, 2H); LCMS: m/z APCI⁺ 268 [MH]⁺

### Preparation 60

### 4-[4-(4-Chlorophenyl)-5-methyl-4H-[1,2,4]triazol-3-yl]-piperidine

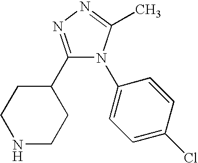

The piperidine of preparation 59 (4.0 g, 15 mmol) was dissolved in toluene (100 ml) and para-chloroaniline (2.1 g, 16.5 mmol) was added, followed by trifluoroacetic acid (2 ml). The solution was heated at 110° C. for 16 hours. Additional trifluoroacetic acid (2 ml) was added, and the solution was heated at 110° C. for a further 48 hours. The reaction mixture was then cooled, an aqueous solution of sodium bicarbonate (75 ml) was added and the organic phase was decanted off. The aqueous phase was basified with potassium carbonate (10 g) and extracted with dichloromethane (4×50 ml). The dichloromethane solution was dried over magnesium sulfate, filtered and the solvent was removed in vacuo to yield the title compound (2.90 g, 70%) as a white solid.

¹H NMR (400 MHz, CDCl₃): δ 1.60-2.00 (m, 2H), 2.20 (s, 3H), 2.40-2.80 (m, 5H), 3.10 (m, 2H), 7.10 (d, 2H), 7.55 (d, 2H); LCMS: m/z APCI⁺ 277 [MH]⁺

### Preparation 61

### 3-Fluoropyridine-2-carbaldehyde

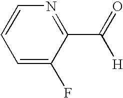

n-Butyllithium (2.0 M in hexanes, 27.5 ml, 55 mmol) was added dropwise over 10 minutes at −20° C. to a solution of N,N,N′,N′-tetramethylethylenediamine (7.5 ml, 50 mmol) in anhydrous diethyl ether (200 ml). The reaction mixture was stirred at −20° C. for 1 hour, then it was cooled to −78° C. and 3-fluoropyridine (4.3 ml, 50 mmol), in diethyl ether (10 ml), was added dropwise over 15 minutes at −78° C. The reaction mixture was stirred at −78° C. for 1 hour, after which time N,N-Dimethylformamide (4.3 ml, 55 mmol), in diethyl ether (10 ml), was added dropwise over 10 minutes at −78° C. It was stirred for 2 hours, then poured carefully onto a rapidly stirring ice/water mixture (300 ml). The mixture was stirred for 20 minutes, then diluted with ethyl acetate (200 ml). The layers were separated and the aqueous layer was further extracted with dichloromethane (4×50 ml). The organic solutions were combined and concentrated under reduced pressure, and the resulting crude product was purified by column chromatography on silica gel using dichloromethane:pentane as eluant (0:100 to 60:40 v/v) to yield the title compound (2.7 g, 43%) as a solid.

¹H NMR (400 MHz, CDCl₃): δ 7.56-7.60 (m, 2H), 8.63 (m, 1H), 10.22 (s, 1H).

### Preparation 62

### 3-Fluoropyridine-2-carbaldehyde oxime

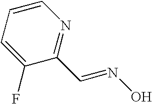

Solid sodium hydroxide (1.08 g, 27 mmol) was added to a solution of hydroxylamine hydrochloride (1.9 g, 27 mmol), in ethanol (120 ml), and the mixture was stirred at room temperature for 30 minutes. The aldehyde of preparation 61 (2.7 g, 22 mmol) was added and the reaction mixture was stirred at room temperature for 6.5 hours. The reaction mixture was then concentrated under reduced pressure and the residue was taken up in dichloromethane (50 ml), and washed with water (50 ml). The organic layer was separated and concentrated under reduced pressure to yield the title compound (2.79 g, 90%) as an off-white solid.

¹H NMR (400 MHz, CDCl₃): δ 7.35 (m, 1H), 7.50 (t, 1H), 8.43 (s, 1H), 8.50 (d, 1H).

### Preparation 63

### 3-Fluoro-N-hydroxypyridine-2-carboximidoyl chloride

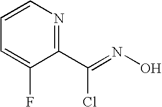

The oxime of preparation 62 (200 mg, 1.4 mmol) was suspended in chloroform (1.5 ml), and then pyridine (11 μl, 0.14 mmol) was added. The reaction mixture was warmed to 40° C. and N-chlorosuccinimide (206 mg, 1.54 mmol) was added. It was then stirred at 40° C. for 3 hours, after which time it was diluted with dichloromethane (50 ml) and washed 3 times with water (3×30 ml). The organic layer was concentrated under reduced pressure to yield the title compound (59 mg, 25%) as a solid.

¹H NMR (400 MHz, CDCl₃): δ 7.43 (m, 1H), 7.55 (t, 1H), 8.56 (d, 1H), 8.75 (brs, 1H).

### Preparation 64

### (E/Z)-1-{4-[4-(4-Chlorophenyl)-5-methyl-4H-[1,2,4]triazol-3-yl]-piperidin-1-yl}-1-(3-fluoropyridin-2-yl)-N-hydroxymethanimine

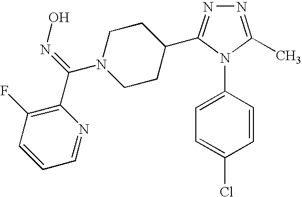

The imidoyl chloride of preparation 63 (59 mg, 0.33 mmol) was added to a solution of the piperidine of preparation 60 (140 mg, 0.5 mmol) and triethylamine (138 μl, 0.99 mmol) in dichloromethane (3 ml). The reaction mixture was then stirred at room temperature for 18 hours, after which time it was diluted with dichloromethane (30 ml) and washed with water (30 ml). The aqueous layer was extracted with dichloromethane (3×20 ml), basified with 1M sodium hydroxide (10 ml) and extracted again with dichloromethane (2×30 ml). The organic extracts were combined and concentrated under reduced pressure to give a solid. This was triturated with dichloromethane, the solid was filtered off and dried to yield the title compound (113 mg).

¹H NMR (400 MHz, CDCl₃): δ 1.72-1.76 (m, 2H), 1.95-2.05 (m, 2H), 2.26 (s, 3H), 2.66 (m, 1H), 2.71-2.78 (m, 2H), 3.50-3.55 (m, 2H), 7.19 (d, 2H), 7.38 (m, 1H), 7.48 (t, 1H), 7.55 (d, 2H), 8.52 (d, 1H); LRMS APCI⁺ m/z 415 [MH]⁺.

### Preparation 65

### tert-Butyl 4-[5-(methoxymethyl)-1,3,4-oxadiazol-2-yl]piperidine-1-carboxylate

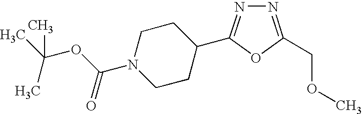

Potassium tert-butoxide (3.40 g, 30.3 mmol) was added to a solution of 4-(5-chloromethyl-[1,3,4]oxadiazol-2-yl)-piperidine-1-carboxylic acid tert-butyl ester (described in WO 2004/037807 prep 74; 7.62 g, 25.25 mmol), in methanol (120 ml), and the reaction mixture was stirred at room temperature for 18 hours. Tlc analysis showed starting material remained, so additional potassium tert-butoxide (1 g, 8.9 mmol) was added, and the reaction mixture was stirred at 50° C. for a further 2 hours. It was then concentrated under reduced pressure, and the residue was partitioned between ethyl acetate (200 ml) and ammonium chloride solution (150 ml). The layers were separated, the organic phase was dried over magnesium sulfate, filtered and evaporated under reduced pressure to afford the title compound (7.3 g, 97%) as a yellow oil.

¹H NMR (400 MHz, CDCl₃): δ 1.45 (s, 9H), 1.82 (m, 2H), 2.08 (m, 2H), 2.96 (m, 2H), 3.08 (m, 1H), 3.44 (s, 3H), 4.10 (m, 2H), 4.61 (s, 2H); LCMS: m/z APCI⁺ 298 [MH]⁺

### Preparation 66

### tert-Butyl 4-[4-(4-chlorophenyl)-5-(methoxymethyl)-4H-1,2,4-triazol-3-yl]piperidine-1-carboxylate

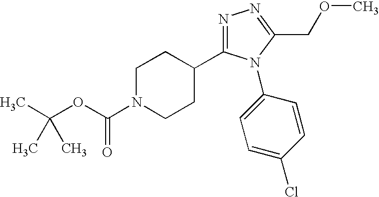

Trifluoroacetic acid (2.14 g, 18.83 mmol) was added to a solution of the piperidine of preparation 65 (7.0 g, 23.54 mmol) and 4-chloroaniline (3.60 g, 28.24 mmol), in toluene (50 ml), and the reaction mixture was heated under reflux for 18 hours. The cooled solution was then concentrated under reduced pressure and the residue was purified by column chromatography using a silica gel cartridge and an elution gradient of dichloromethane:methanol (100:0 to 90:10) to afford the title compound (4.25 g, 44%) as an oil.

¹H NMR (400 MHz, CD₃OD): δ 1.45 (s, 9H), 1.67-1.83 (m, 4H), 2.68-2.83 (m, 3H), 3.32 (s, 3H), 4.08 (m, 2H), 4.39 (s, 2H), 7.46 (d, 2H), 7.63 (d, 2H); LCMS: m/z APCI⁺ 407 [MH]⁺

### Preparation 67

### 4-[4-(4-Chlorophenyl)-5-(methoxymethyl)-4H-1,2,4-triazol-3-yl]piperidine

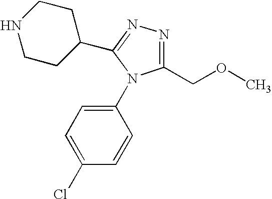

4M Hydrochloric acid in dioxan (60 ml) was added to a solution of the piperidine of preparation 66 (3.75 g, 9.22 mmol), in dioxan (50 ml), and the reaction was stirred at room temperature for 3 hours. It was then evaporated under reduced pressure, and the residue was re-dissolved in dichloromethane (100 ml) and washed with aqueous ammonia (100 ml) and brine (100 ml). The organic solution was dried over magnesium sulfate, filtered and evaporated under reduced pressure. The crude product was purified by column chromatography using a silica gel cartridge and dichloromethane:methanol:0.88 ammonia (90:10:1) as eluant to afford the title compound (1.99 g, 70%).

¹H NMR (400 MHz, CDCl₃): δ 1.80-1.98 (m, 4H), 2.57-2.70 (m, 3H), 3.20 (m, 2H), 3.25 (s, 3H), 4.38 (s, 2H), 7.22 (d, 2H), 7.57 (d, 2H); LCMS: m/z APCI⁺ 307 [MH]⁺

### Preparation 68

### [1,2,3]Triazol-1-yl-acetic acid ethyl ester and [1,2,3]triazol-2-yl-acetic acid ethyl ester

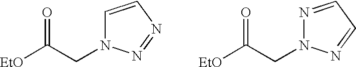

1,2,3-Triazole (19.00 kg, 275 mol) was charged over 30 minutes to a suspension of potassium carbonate (42.15 kg, 305 mol) in ethanol (80 L) and was rinsed in with ethanol (2 L). A solution of ethyl bromoacetate (45.8 kg, 274 mol) in ethanol (30 L) was added slowly and was rinsed in with ethanol (2 L). During this time the reaction temperature was maintained at <20° C. The reaction mixture was then warmed to room temperature and stirred overnight. The suspension was filtered; the residue was washed with ethanol (25 L and 17 L) and then the filtrate was concentrated under reduced pressure. The concentrate was dissolved in ethyl acetate (120 L) and the solution was washed with 1N hydrochloric acid (1×40 L, 7×20 L, 4×15 L). The aqueous washings were combined and extracted with ethyl acetate (3×21 L). The organic phases were combined, dried over magnesium sulfate, filtered and concentrated to dryness giving a mixture of the title compounds (25 kg).

¹H NMR spectroscopic analysis indicated a 6:5 mixture of N-2/N-1 isomers.

¹H NMR (400 MHz, CDCl₃): δ 1.25 (m, 3H), 4.13 (q, 2H, N-1 isomer), 4.25 (q, 2H, N-2 isomer), 5.20 (s, 2H, N-1 isomer), 5.22 (s, 2H, N-2 isomer), 7.70 (s, 2H, N-2 isomer), 7.77 (s, 2H, N-1 isomer).

### Preparation 69

### [1,2,3]Triazol-2-yl-acetic acid hydrazide

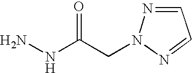

Hydrazine hydrate (8.65 kg, 270 mol) was added to a cooled (<10° C.) solution of the mixture of esters from preparation 68 (19 kg), in ethanol (69 L), keeping the temperature to below 20° C. throughout the addition. The reaction mixture was stirred at between 14 to 19° C. for 3 hours, then more ethanol (25 L) was added and the product was collected by filtration, washing with ethanol (10 L). The crude solid was purified by recrystallisation from ethanol (120 L), followed by three recrystallisations from methanol (105 L, 120 L and 90 L) to yield the title compound (4.53 kg, 12%) after drying in vacuo.

¹H NMR (400 MHz, DMSO-d₆): δ 4.33 (s, 2H), 5.02 (s, 2H), 7.77 (s, 2H), 9.40 (s, 1H).

### EXAMPLES 1 TO 12

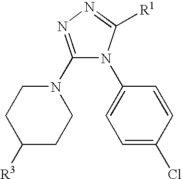

The appropriate hydrazide, R¹CONHNH₂, (1.5 eq.) was added to a solution of the appropriate thiomethyl compound from preparations 31 to 40 (1 eq.), in tetrahydrofuran (7.5 to 18.8 mlmmol⁻¹). Trifluoroacetic acid (0.5 eq.) was added and the reaction mixture was heated at reflux for 1.5 to 3.0 hours. The mixture was concentrated under reduced pressure and the residue was partitioned between ethyl acetate and 2M sodium hydroxide. The aqueous phase was extracted again with ethyl acetate and the organic solutions were combined, washed with brine, dried over magnesium sulfate, filtered and concentrated under reduced pressure. The crude product was purified by column chromatography on silica gel using dichloromethane/methanol/aqueous ammonia as eluant (95:5:0.5 v/v/v) to yield the title compound.

| Ex no | Data |
|---|---|
| 1 | R¹ = CH₃; R³ = 2-methyl-1H-benzimidazol-1-yl |
| | ¹H NMR (400 MHz, CDCl₃): δ 1.78-1.82 (m, 2H), 2.28 (s, 3H), 2.46-2.56 (m, 2H), 2.62 |
| | (s, 3H), 3.01 (t, 2H), 3.49-3.52 (m, 2H), 4.28 (m, 1H), 7.20-7.23 (m, 2H), 7.32 (d, 2H), |
| | 7.43 (m, 1H), 7.56 (d, 2H), 7.68 (m, 1H); LRMS APCI⁺ m/z 407 [MH]⁺; Microanalysis: |
| | Found; C, 62.90; H, 5.60; N, 19.81; C₂₂H₂₃N₆Cl•0.2DCM requires; C, 62.90; H, 5.56; |
| | N, 19.83%; 36% yield. |
| 2 | R¹ = CH₃; R³ = 3-methyl-2-oxo-2,3-dihydro-1H-benzimidazol-1-yl |
| | ¹H NMR (400 MHz, CDCl₃): δ 1.72 (d, 2H), 2.26 (s, 3H), 2.38-2.48 (m, 2H), 2.98 (t, |
| | 2H), 3.39 (s, 3H), 3.43-3.47 (m, 2H), 4.37 (m, 1H), 6.97 (m, 1H), 7.07-7.11 (m, 3H), |
| | 7.31 (d, 2H), 7.55 (d, 2H); LRMS APCI⁺ m/z 423 [MH]⁺; 31% yield |
| 3 | R¹ = CH₃; R³ = 2-oxo-2,3-dihydro-1H-benzimidazol-1-yl |
| | ¹H NMR (400 MHz, CD₃OD): δ 1.66 (d, 2H), 2.22 (s, 3H), 2.35-2.46 (m, 2H), 2.96 (t, |
| | 2H), 3.42 (m, 2H), 4.32 (m, 1H), 7.01-7.05 (m, 3H), 7.16 (m, 1H), 7.53 (d, 2H), 7.64 |
| | (d, 2H); LRMS APCI⁺ m/z 409 [MH]⁺; 21% yield |
| 4 | R¹ = CH₃; R³ = 5-fluoro-2-oxo-2,3-dihydro-1H-benzimidazol-1-yl |
| | ¹H NMR (400 MHz, DMSO-D₆): δ 1.60 (d, 2H), 2.19 (s, 3H), 2.23-2.30 (m, 2H), 3.09 (t, |
| | 2H), 3.37-3.41 (m, 2H), 4.32 (m, 1H), 6.79-6.83 (m, 2H), 7.13 (m, 1H), 7.73-7.76 (m, |
| | 4H), 11.03 (s, 1H); LRMS APCI⁺ m/z 427 [MH]⁺; 19% yield |
| 5 | R¹ = CH₃; R³ = 5-chloro-2-oxo-2,3-dihydro-1H-benzimidazol-1-yl |
| | ¹H NMR (400 MHz, CD₃OD): δ 1.70 (d, 2H), 2.24 (s, 3H), 2.34-2.44 (m, 2H), 2.98 (t, |
| | 2H), 3.43 (d, 2H), 4.32 (m, 1H), 7.04-7.07 (m, 2H), 7.16 (d, 1H), 7.55 (d, 2H), 7.66 (d, |
| | 2H) LRMS ESI⁺m/z 465 [MNa]⁺; 14% yield |
| 6^{A} | R¹ = CH₃; R³ = 2-oxo[1,3]oxazolo[4,5-b]pyridin-3(2H)-yl |
| | ¹H NMR (400 MHz, CDCl₃): δ 1.76 (d, 2H), 2.23 (s, 3H), 2.46-2.56 (m, 2H), 2.93 (t, |
| | 2H), 3.38-3.43 (m, 2H), 4.34 (m, 1H), 7.02 (m, 1H), 7.31 (d, 2H), 7.37 (d, 1H), 7.52 (d, |
| | 2H), 8.08 (m, 1H). LRMS APCI⁺ m/z 411 [MH]⁺. 37% yield |
| 7^{A} | R¹ = [1,2,3]-triazol-2-ylmethyl; R³ = 2-oxo[1,3]oxazolo[4,5-b]pyridin-3(2H)-yl |
| | ¹H NMR (400 MHz, CD₃OD): δ 1.75-1.79 (m, 2H), 2.45-2.55 (m, 2H), 2.96-3.03 (m, |
| | 2H), 3.46-3.50 (m, 2H), 4.39 (m, 1H), 5.68 (s, 2H), 7.12 (m, 1H), 7.36 (d, 2H), 7.51- |
| | 7.54 (m, 3H), 7.57 (s, 2H), 8.08 (m, 1H); LRMS APCI⁺ m/z 478 [MH]⁺; 37% yield. |
| 8^{A} | R¹ = CH₃; R³ = 3-oxo-2,3-dihydro-4H-pyrido[3,2-b][1,4]oxazine-4-yl |
| | ¹H NMR (400 MHz, CDCl₃): δ 1.55-1.61 (m, 2H), 2.23 (s, 3H), 2.63-2.72 (m, 2H), 2.87- |
| | 2.94 (m, 2H), 3.35-3.39 (m, 2H), 4.53 (s, 2H), 4.97 (m, 1H), 6.93 (m, 1H), 7.20 (d, |
| | 1H), 7.31 (d, 2H), 7.52 (d, 2H), 7.99 (m, 1H); LRMS APCl⁺m/z 425 [MH]⁺; 27% yield. |
| 9^{B} | R¹ = CH₃; R³ = 1H-1,2,3-benzotriazol-1-yl. |
| | ¹H NMR (400 MHz, CDCl₃): δ 2.07-2.10 (m, 2H), 2.24 (s, 3H), 2.32-3.37 (m, 2H), 3.07 |
| | (t, 2H), 3.49 (d, 2H), 4.72 (m, 1H), 7.19-7.29 (m, 3H), 7.46 (t, 1H), 7.50-7.54 (m, 3H), |
| | 8.01 (d, 1H); LRMS APCI⁺ m/z 394 [MH]⁺; 44% yield. |
| 10^{C} | R¹ = CH₃; R³ = 3-isopropyl-1,2,4-oxadiazol-5-yl |
| | ¹H NMR (400 MHz, CDCl₃): δ 1.31 (d, 6H), 1.76-1.86 (m, 2H), 2.00-2.07 (m, 2H), 2.27 |
| | (s, 3H), 2.92-3.08 (m, 4H), 3.35-3.40 (m, 2H), 7.31 (d, 2H), 7.54 (d, 2H); LRMS APCI⁺ |
| | m/z 387 [MH]⁺; 65% yield |
| 11 | R¹ = CH₃; R³ = 5-isopropyl-1,2,4-oxadiazol-3-yl |
| | ¹H NMR (400 MHz, CDCl₃): δ 1.35 (d, 6H), 1.67-1.77 (m, 2H), 1.92-1.97 (m, 2H), 2.23 |
| | (s, 3H), 2.80-2.93 (m, 3H), 3.15 (m, 1H), 3.32-3.36 (m, 2H), 7.26 (d, 2H), 7.50 (d, 2H); |
| | LCMS ELSD-APCI⁺ single peak m/z 387 [MH]⁺; 25% yield. |
| 12 | R¹ = [1,2,3]-triazol-2-ylmethyl; R³ = 5-isopropyl-1,2,4-oxadiazol-3-yl |
| | ¹H NMR (400 MHz, CDCl₃): δ 1.31 (d, 6H), 1.61-1.71 (m, 2H), 1.86-1.92 (m, 2H), 2.76- |
| | 2.91 (m, 3H), 3.12 (m, 1H), 3.31-3.35 (m, 2H), 5.54 (s, 2H), 7.08 (d, 2H), 7.35 (d, 2H), |
| | 7.46 (s, 2H); LRMS APCI⁺ m/z 454 [MH]⁺. |

A = 1.5 equivalents of trifluoroacetic acid were used.
B = 2 eq. of hydrazide were used
C = 1.1 eq of hydrazide were used

### EXAMPLE 13

### 3-{1-[4-(4-Chlorophenyl)-5-methyl-4H-1,2,4-triazol-3-yl]piperidin-4-yl}[1,2,4]triazolo[4,3-b]pyridazine

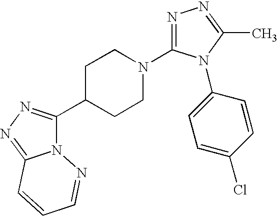

Potassium tert-butoxide (75 mg, 0.67 mmol) was added to a solution of the thioamide of preparation 25 (250 mg, 0.67 mmol) in tetrahydrofuran (5 ml), and the solution was stirred for 10 minutes. Methyl tosylate (125 mg, 0.67 mmol) was then added and the reaction mixture was stirred at room temperature for a further 30 minutes. Acetic hydrazide (60 mg, 0.8 mmol) was then added, followed by trifluoroacetic acid (25 μl, 0.34 mmol). The reaction mixture was heated at reflux for 2 hours, after which time, it was cooled, then aqueous ammonia (10 drops) was added along with methanol (20 ml) and silica (5 g), and the mixture was concentrated under reduced pressure. The residue containing the crude product was azeotroped with dichloromethane and loaded onto a silica gel column. The crude product was purified by column chromatography on silica gel using dichloromethane/methanol/aqueous ammonia as eluant (93:7:1 v/v/v). The product was partitioned between ethyl acetate (200 ml) and 2M sodium hydroxide solution (50 ml). The organic phase was washed with brine (50 ml), dried over magnesium sulfate, filtered, concentrated under reduced pressure and triturated with diethyl ether to yield the title compound (27 mg, 10%)

¹H NMR (400 MHz, CDCl₃): δ 1.98-2.08 (m, 2H), 2.11-2.16 (m, 2H), 2.28 (s, 3H), 3.03-3.10 (m, 2H), 3.44-3.50 (m, 3H), 7.09 (dd, 1H), 7.33 (d, 2H), 7.54 (d, 2H), 8.10 (d, 1H), 8.33 (m, 1H); LRMS ESI⁺ m/z 417 [MNa]⁺.

### EXAMPLE 14

### 3-{1-[4-(4-Chlorophenyl)-5-methyl-4H-1,2,4-triazol-3-yl]piperidin-4-yl}-1,3-dihydro-2H-imidazo[4,5-b]pyridin-2-one

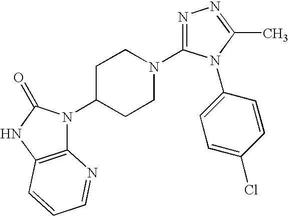

N,N′-Carbonyldiimidazole (63.2 mg, 0.39 mmol) was added to a solution of the pyridine of preparation 51 (150 mg, 0.39 mmol) in tetrahydrofuran (15 ml). The reaction mixture was stirred at room temperature for 18 hours, then heated at reflux for a further 7 hours. Additional N,N′-carbonyldiimidazole (123 mg, 0.80 mmol) was added, and then the reaction mixture was stirred at reflux for 18 hours. The cooled mixture was concentrated under reduced pressure and the residue was partitioned between water (25 ml) and ethyl acetate (25 ml). The organic layer was washed with brine (25 ml), dried over magnesium sulfate, filtered and concentrated under reduced pressure. The crude product was purified by column chromatography on silica gel using dichloromethane/methanol/aqueous ammonia as eluant (95:5:0.5 v/v/v) to yield the title compound (117 mg, 73%) after trituration with diethyl ether.

¹H NMR (400 MHz, CDCl₃): δ 1.72 (d, 2H), 2.25 (s, 3H), 2.62-2.72 (m, 2H), 2.95 (t, 2H), 3.42-3.46 (m, 2H), 4.48 (m, 1H), 6.95 (m, 1H), 7.27 (m, 1H), 7.33 (d, 2H), 7.53 (d, 2H), 7.70 (s, 1H), 7.99 (m, 1H); LRMS APCI⁺ m/z 410 [MH]⁺.

### EXAMPLE 15

### 3-{1-[4-(4-Chlorophenyl)-5-methyl-4H-1,2,4-triazol-3-yl]piperidin-4-yl}-3H-imidazo[4,5-b]pyridine

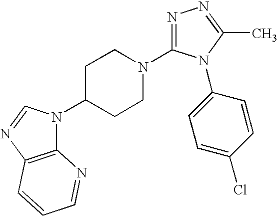

The pyridine of preparation 51 (200 mg, 0.52 mmol) was heated at reflux in formic acid (2 ml) for 14 hours. The cooled mixture was concentrated under reduced pressure and the residue was partitioned between dilute aqueous ammonia (25 ml) and ethyl acetate (25 ml). The organic layer was washed with brine (25 ml), dried over magnesium sulfate, filtered and concentrated under reduced pressure. The crude product was purified by column chromatography on silica gel using dichloromethane/methanol/aqueous ammonia as eluant (95:5:0.5 v/v/v) to yield the title compound (90 mg, 44%) after trituration with diethyl ether.

¹H NMR (400 MHz, CDCl₃): δ 2.08-2.18 (m, 4H), 2.27 (s, 3H), 3.07-3.13 (m, 2H), 3.49-3.53 (m, 2H), 4.72 (m, 1H), 7.24 (m, 1H), 7.32 (d, 2H), 7.56 (d, 2H), 8.07 (d, 1H), 8.13 (s, 1H), 8.38 (m, 1H); LRMS ESI⁺ m/z 416 [MNa]⁺.

### EXAMPLE 16

### 3-{1-[4-(4-Chlorophenyl)-5-methyl-4H-1,2,4-triazol-3-yl]piperidin-4-yl}-3H-[1,2,3]triazolo[4,5-b]pyridine

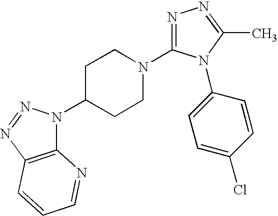

Sodium nitrite (61 mg, 0.87 mmol) in water (1 ml) was added dropwise at 0° C. to a solution of the pyridine of preparation 51 (300 mg, 0.78 mmol) in 2N hydrochloric acid (4 ml). The reaction mixture was stirred at 0° C. for 40 minutes, and then dilute aqueous ammonia (10 ml) was added carefully. The resulting precipitate was filtered off and dried in vacuo to yield the title compound (220 mg, 71%).

¹H NMR (400 MHz, CDCl₃): δ 2.16 (m, 2H), 2.26 (s, 3H), 2.36-2.46 (m, 2H), 3.10 (t, 2H), 3.51 (d, 2H), 5.04 (m, 1H), 7.31-7.37 (m, 3H), 7.55 (d, 2H), 8.38 (d, 1H), 8.64 (d, 1H); LRMS ESI⁺ m/z 417 [MNa]⁺.

### EXAMPLE 17

### 1-{1-[4-(4-Chlorophenyl)-5-methyl-4H-1,2,4-triazol-3-yl]piperidin-4-yl}-1H-benzimidazol-2-amine

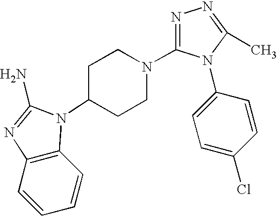

Cyanogen bromide (118 mg, 1.11 mmol) was added to a solution of the amine of preparation 53 (300 mg, 0.78 mmol) in tetrahydrofuran (10 ml). The reaction mixture was heated at reflux for 66 hours, then allowed to cool. Water (5 ml) and 2M sodium hydroxide (2 ml, 4 mmol) were added and the mixture was stirred at room temperature for 1 hour. The organic solvent was then removed under reduced pressure, causing a solid to precipitate. The aqueous solution was decanted off and the solid was purified by column chromatography on silica gel using dichloromethane/methanol/aqueous ammonia as eluant (90:10:1 v/v/v) to yield the title compound (196 mg, 62%).

¹H NMR (400 MHz, CDCl₃): δ 1.78-1.83 (m, 2H), 2.27 (s, 3H), 2.43-2.53 (m, 2H), 3.03 (t, 2H), 3.44 (d, 2H), 4.22 (m, 1H), 7.06 (t, 1H), 7.13 (t, 1H), 7.22 (d, 1H), 7.31 (d, 2H), 7.42 (d, 1H), 7.56 (d, 2H); LRMS APCI⁺ m/z 408 [MH]⁺; Microanalysis: Found; C, 60.66; H, 5.54; N, 23.30; C₂₁H₂₂ClN₇·0.44H₂O requires; C, 60.66; H, 5.55; N, 23.58%.

### EXAMPLE 18

### 1-{1-[4-(4-Chlorophenyl)-5-methyl-4H-1,2,4-triazol-3-yl]piperidin-4-yl}-3-methyl-1,3-dihydro-2,1,3-benzothiadiazole 2,2-dioxide

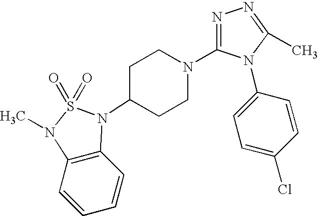

Sodium hydride (16 mg, 60% dispersion in mineral oil, 0.40 mmol) was added at 0° C. to a solution of the benzothiadiazole of preparation 54 (87 mg, 0.20 mmol), in tetrahydrofuran (2 ml) and N,N-dimethylformamide (1 ml). Methyl iodide (12.5 μl, 0.20 mmol) was added after 2 minutes and the reaction mixture was then stirred at 0° C. for 15 minutes. The reaction was incomplete (as assessed by tlc) so additional sodium hydride (7.6 mg, 60% dispersion in mineral oil, 0.19 mmol) was added and the reaction mixture was stirred at room temperature for a further 18 hours. Water (20 ml) was added carefully, which caused effervescence and a solid to precipitate. This solid was filtered off and dried to yield the title compound (65 mg, 71%).

¹H NMR (400 MHz, CD₃OD): δ 1.97-2.01 (m, 2H), 2.13-2.24 (m, 5H), 2.97 (t, 2H), 3.20 (s, 3H), 3.39-3.43 (m, 2H), 4.14 (m, 1H), 6.88 (m, 1H), 6.95-7.00 (m, 3H), 7.52 (d, 2H), 7.65 (d, 2H); LRMS ESI⁺ m/z 481 [MNa]⁺; Mp=210 to 212° C.

### EXAMPLE 19

### 3-{1-[4-(4-Chlorophenyl)-5-methyl)-4H-1,2,4-triazol-3-yl]piperidin-4-yl}-6-fluoro-3H-[1,2,3]triazolo[4,5-b]pyridine

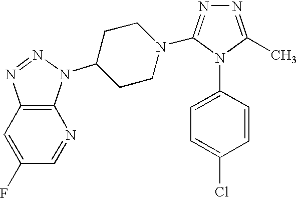

Sodium nitrite (15 mg, 0.20 mmol), in water (1 ml), was added dropwise at 0° C. to a solution of the pyridine of preparation 58 (90 mg, 0.19 mmol), in 2N hydrochloric acid (1 ml). The reaction mixture was stirred at 0° C. for 1 hour, after which time, dilute aqueous ammonia was added carefully, which caused a solid to precipitate. This solid was filtered off and dissolved in dichloromethane (25 ml). The solution was washed with water (2×25 ml) and the combined aqueous layers were extracted with dichloromethane (25 ml). The combined organic solutions were dried over magnesium sulfate, filtered and concentrated under reduced pressure. The crude product was purified by column chromatography on silica gel using dichloromethane/methanol/aqueous ammonia as eluant (100:0:0 to 95:5:0.5 v/v/v) to yield the title compound (31 mg, 40%).

¹H NMR (400 MHz, CDCl₃): δ 2.12-2.18 (m, 2H), 2.27 (s, 3H), 2.34-2.45 (m, 2H), 3.09 (t, 2H), 3.48-3.53 (m, 2H), 5.01 (m, 1H), 7.32 (d, 2H), 7.55 (d, 2H), 8.00 (dd, 1H), 8.56 (m, 1H); LRMS APCI⁺ m/z 413 [MH]⁺.

### EXAMPLE 20

### 3-{4-[4-(4-Chlorophenyl)-5-methyl-4H-1,2,4-triazol-3-yl]piperazin-1-yl}-1,2-benzisothiazole

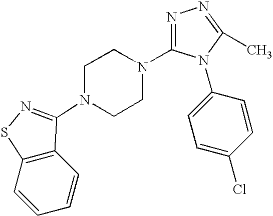

The title compound (106 mg, 51%) was prepared by a method similar to that described for examples 1 to 12 using the compound of preparation 43 and acetic hydrazide.

¹H NMR (400 MHz, CDCl₃): δ 2.23 (s, 3H), 3.23-3.26 (m, 4H), 3.38-3.41 (m, 4H), 7.25-7.33 (m, 3H), 7.42 (t, 1H), 7.49 (d, 2H), 7.76 (d, 1H), 7.81 (d, 1H); LRMS APCI⁺ m/z 411 [MH]⁺.

### EXAMPLE 21

### 3-{4-[4-(4-Chlorophenyl)-5-methyl-4H-1,2,4-triazol-3-yl]piperazin-1-yl}-1,2-benzisothiazole 1,1-dioxide

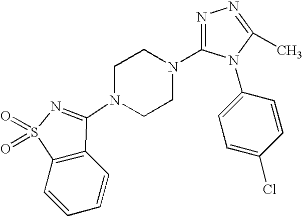

Metachloroperbenzoic acid (74.4 mg, 0.43 mmol) was added to a solution of the benzisothiazole of example 20 (80.5 mg, 0.19 mmol) in dichloromethane (4 ml). The reaction mixture was stirred at room temperature for 2 hours. It was then diluted with dichloromethane (15 ml), washed with 1M sodium hydroxide (10 ml), dried over magnesium sulfate, filtered and concentrated under reduced pressure. The residue was taken up in hot ethyl acetate (10 ml) and solidified upon cooling. The solvent was removed under reduced pressure and the solid crystallised from ethanol. This was filtered and rinsed with cold ethanol to yield the title compound (48.6 mg, 56%).

¹H NMR (400 MHz, CDCl₃): δ 2.26 (s, 3H), 3.26-3.34 (m, 4H), 3.96-4.06 (m, 4H), 7.29 (d, 2H), 7.56 (d, 2H), 7.64 (t, 1H), 7.71 (t, 1H), 7.79 (d, 1H), 7.95 (d, 1H); LCMS UV-ELSD-ESI⁺ single peak m/z 443 [MH]⁺.

### EXAMPLE 22

### 3-{4-[4-(4-Chlorophenyl)-5-(trifluoromethyl)-4H-1,2,4-triazol-3-yl]piperazin-1-yl}-1,2-benzisothiazole

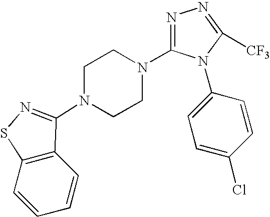

The title compound (150 mg, 16%) was prepared by a method similar to that described for examples 1 to 12 using the compound of preparation 43 and trifluoroacetic acid hydrazide.

¹H NMR (400 MHz, CDCl₃): δ 3.33-3.36 (m, 4H), 3.41-3.45 (m, 4H), 7.32-7.36 (m, 3H), 7.46 (t, 1H), 7.53 (d, 2H), 7.78-7.84 (m, 2H); LRMS APCI⁺ m/z 465 [MH]⁺.

### EXAMPLE 23

### 3-{4-[4-(4-Chlorophenyl)-5-(trifluoromethyl)-4H-1,2,4-triazol-3-yl]piperazin-1-yl}-1,2-benzisothiazole 1,1-dioxide

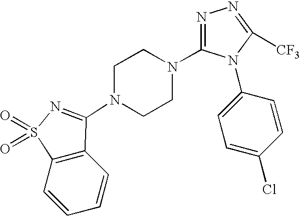

The title compound (34 mg, 57%) was prepared by a method similar to that described for example 21 using the benzisothiazole of example 22 and metachloroperbenzoic acid.

¹H NMR (400 MHz, CDCl₃): δ 3.33-3.43 (m, 4H), 4.00-4.07 (m, 4H), 7.35 (d, 2H), 7.58 (d, 2H), 7.65 (t, 1H), 7.73 (t, 1H), 7.77 (d, 1H), 7.96 (d, 1H); LCMS ELSD-APCI⁺ single peak m/z 497 [MH]⁺.

### EXAMPLE 24

### 3-{(3-endo)-8-[4-(4-Chlorophenyl)-5-methyl-4H-1,2,4-triazol-3-yl]-8-azabicyclo[3.2.1]oct-3-yl}-2-methyl-3H-imidazo[4,5-c]pyridine

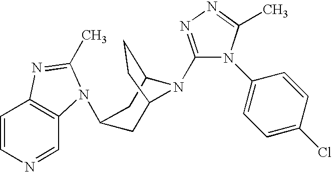

The title compound (567 mg, 56%) was prepared by a method similar to that described for examples 1 to 12 using the compound of preparation 47 and acetic hydrazide.

¹H NMR (400 MHz, CDCl₃): δ 1.83-1.88 (m, 2H), 1.97-2.03 (m, 2H), 2.25 (s, 3H), 2.27-2.32 (m, 2H), 2.35-2.42 (m, 2H), 2.57 (s, 3H), 4.01-4.04 (m, 2H), 4.62 (m, 1H), 7.33 (d, 2H), 7.54-7.58 (m, 3H), 8.36 (d, 1H), 8.77 (s, 1H); LRMS APCI⁺ m/z 434 [MH]⁺.

### EXAMPLE 25

### 3-{4-[4-(4-Chlorophenyl)-5-methyl-4H-1,2,4-triazol-3-yl]piperidin-1-yl}-1,2-benzisothiazole

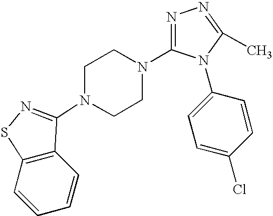

3-Chloro-1,2-benzisothiazole (147 mg, 0.87 mmol) was added to a solution of the piperidine of preparation 60 (200 mg, 0.72 mmol) in acetonitrile (20 ml). 1,8-Diazabicyclo[5.4.0]undec-7-ene (111 μl, 0.72 mmol) was added, and the reaction mixture was then stirred at room temperature for 48 hours. It was then concentrated under reduced pressure and the crude product was purified by column chromatography on silica gel using dichloromethane/methanol/aqueous ammonia as eluant (90:10:1 v/v/v) to yield the title compound (290 mg, 98%) as a solid.

¹H NMR (400 MHz, CD₃OD): δ 1.80-1.85 (m, 2H), 2.00-2.10 (m, 2H), 2.23 (s, 3H), 2.57 (m, 1H), 2.85-2.91 (m, 2H), 3.23-3.28 (m, 2H), 7.32 (t, 1H), 7.43 (d, 2H), 7.61-7.68 (m, 4H), 7.74 (d, 1H); LRMS APCI⁺ m/z 410 [MH]⁺.

### EXAMPLE 26

### 3-{4-[4-(4-Chlorophenyl)-5-(methoxymethyl)-4H-1,2,4-triazol-3-yl]piperidin-1-yl}-1,2-benzisothiazole

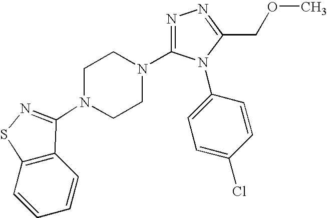

The title compound (40 mg, 25%) was prepared by a method similar to that described for example using the piperidine of preparation 60 and 3-chloro-1,2-benzisothiazole, (except that 1.1 eq. of 1,8-diazabicyclo[5.4.0]undec-7-ene were used).

¹H NMR (400 MHz, CDCl₃): δ 1.79-1.85 (m, 2H), 2.16-2.24 (m, 2H), 2.49 (m, 1H), 2.84 (m, 2H), 3.30 (m, 5H), 4.38 (s, 2H), 7.12 (m, 4H), 7.52-7.61 (m, 3H), 7.70 (d, 1H); LRMS APCI⁺ m/z 440 [MH]⁺.

### EXAMPLE 27

### 3-{4-[4-(4-Chlorophenyl)-5-methyl-4H-1,2,4-triazol-3-yl]piperidin-1-yl}isothiazolo[5,4-b]pyridine

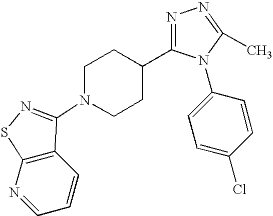

The title compound was prepared by a method similar to that described for example 25 using the piperidine of preparation 60 and 3-chloroisothiazolo[5,4-b]pyridine.

¹H NMR (400 MHz, CDCl₃): δ 1.75 (m, 2H), 1.98-2.10 (m, 2H), 2.21 (s, 3H), 2.54 (m, 1H), 3.25 (m, 2H), 3.48-3.68 (m, 2H), 7.02 (m, 1H), 7.18 (d, 2H), 7.57 (d, 2H), 7.78 (dd, 1H), 8.50 (d, 1H); LCMS APCI⁺ m/z 411 [MH]⁺.

### EXAMPLE 28

### 3-{4-[4-(4-Chlorophenyl)-5-methyl-4H-1,2,4-triazol-3-yl]piperidin-1-yl}-1,2-benzisothiazole 1,1-dioxide

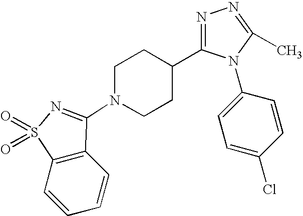

The title compound (36 mg, 42%) was prepared by a method similar to that described for example 521 using the benzisothiazole of example 25 and metachloroperbenzoic acid.

¹H NMR (400 MHz, CDCl₃): δ 1.82-1.88 (m, 2H), 1.93-2.02 (m, 2H), 2.21 (s, 3H), 2.57 (m, 1H), 2.78-2.84 (m, 2H), 3.81-3.86 (m, 2H), 7.12 (d, 2H), 7.54 (d, 2H), 7.66-7.76 (m, 2H), 7.86 (d, 1H), 8.02 (d, 1H); LRMS APCI⁺ m/z 442 [MH]⁺.

### EXAMPLE 29

### 3-{4-[4-(4-Chlorophenyl)-5-methyl-4H-1,2,4-triazol-3-yl]piperidin-1-yl}isoxazolo[4,5-b]pyridine

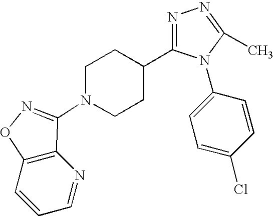

Sodium hydride (60% dispersion in oil, 19 mg, 0.48 mmol) was added to a solution of the compound of preparation 64 (180 mg, 0.43 mmol), in tetrahydrofuran (1 ml), and the reaction mixture was stirred for 5 minutes at room temperature. Toluene (4 ml) was added and the reaction mixture was then heated at 110° C. for 18 hours. Ethanol (74 μl, 1.3 mmol) was added to the cooled mixture, followed by acetic acid (18 μl, 0.3 mmol). The reaction mixture was stirred at room temperature for 20 minutes, then diluted with dichloromethane (15 ml) and washed with water (10 ml). The layers were separated and the aqueous layer was extracted further with dichloromethane (3×10 ml). The aqueous solution was basified using 1M sodium hydroxide (10 ml) and extracted again with dichloromethane (2×10 ml). The combined organic solutions were concentrated under reduced pressure, and the resulting crude product was purified by column chromatography on silica gel using dichloromethane/methanol/aqueous ammonia as eluant (100:0:0 to 95:5:0.5 v/v/v) to yield the title compound (45 mg, 27%).

¹H NMR (400 MHz, CDCl₃): δ 1.89 (d, 2H), 2.06-2.17 (m, 2H), 2.24 (s, 3H), 2.74 (m, 1H), 3.03 (t, 2H), 4.72 (d, 2H), 7.19 (d, 2H), 7.36 (m, 1H), 7.56 (d, 2H), 7.70 (d, 1H), 8.50 (d, 1H); LCMS UV-ELSD-ESI⁺ single peak m/z 395 [MH]⁺.

All of the compounds exemplified above showed a Ki value of less than 700 nM when tested in screen 1.0 (V_{1A} filter binding assay) as described above.

Examples of specific compounds are illustrated below:

| Example No. | Ki (nM) |
|---|---|
| 7 | 1.10 |
| 20 | 0.49 |
| 22 | 0.79 |
| 29 | 1.04 |

1.-21. (canceled)

22. A compound of formula (I), 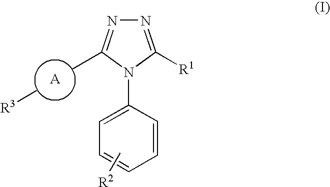
or a pharmaceutically acceptable salt thereof, wherein:
R¹ is a group selected from H, trifluoromethyl, and C₁₋₆ alkyl, wherein said C₁₋₆ alkyl is optionally substituted by C₁₋₆ alkyloxy or triazolyl;
R² is halo;
Ring A is a 5- or 6-membered heterocyclic ring containing one to three N atoms and wherein the ring is optionally bridged with two to five carbon atoms; and
R³ is a 5- or 6-membered heterocyclic ring containing one to four atoms selected from N, O and S, the heterocyclic ring being optionally substituted by one to four groups selected from C₁₋₆ alkyl, oxo and NH₂, the heterocyclic ring being further optionally fused to a 5- or 6-membered aryl or heterocyclic ring containing one to four atoms selected from N, O and S, the fused aryl or heterocyclic ring being optionally substituted by one to four halo atoms.

23. The compound of claim 22, wherein R¹ is methyl, trifluoromethyl, methoxymethyl, or triazolyl-methyl; or a pharmaceutically acceptable salt thereof.

24. The compound of claim 23, wherein R² is chloro; or a pharmaceutically acceptable salt thereof.

25. The compound of claim 22, wherein ring A is piperidinyl or piperazinyl; or a pharmaceutically acceptable salt thereof.

26. The compound of claim 22, wherein R³ is a 5- or 6-membered heterocyclic ring containing one to four atoms selected from N, O or S, the heterocyclic ring being optionally substituted by one to four groups selected from C₁₋₆ alkyl, oxo and NH₂, the heterocyclic ring being fused to a 5- or 6-membered aryl or heterocyclic ring containing one to four atoms selected from N, O or S, the fused aryl or heterocyclic ring being substituted by one to two halo atoms; or a pharmaceutically acceptable salt thereof.

27. The compound of claim 26, wherein R¹ is a 5- or 6-membered heterocyclic ring containing one to four atoms selected from N, O or S, the heterocyclic ring being optionally substituted by one to four groups selected from C₁₋₆ alkyl, oxo and NH₂, the heterocyclic ring being fused to a phenyl or pyridyl ring, the phenyl or pyridyl ring being substituted by one to two halo atoms; or a pharmaceutically acceptable salt thereof.

28. The compound of 27, wherein R³ is: 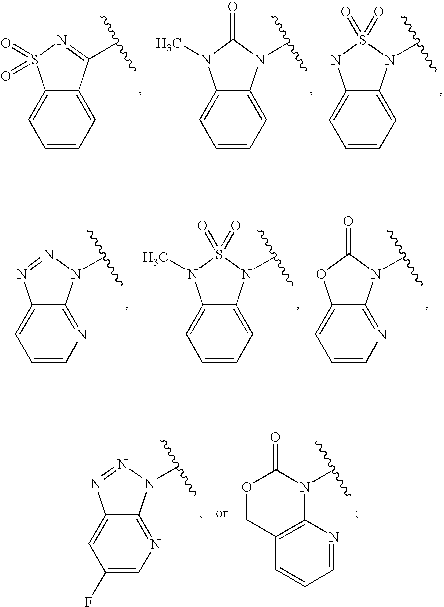
or a pharmaceutically acceptable salt thereof.

29. The compound of claim 22 selected from
1-{1-[4-(4-chlorophenyl)-5-methyl-4H-1,2,4-triazol-3-yl]piperidin-4-yl}-2-methyl-1H-benzimidazole;
1-{1-[4-(4-chlorophenyl)-5-methyl-4H-1,2,4-triazol-3-yl]piperidin-4-yl}-1,3-dihydro-2H-benzimidazol-2-one;
1-{1-[4-(4-chlorophenyl)-5-methyl-4H-1,2,4-triazol-3-yl]piperidin-4-yl}-3-methyl-1,3-dihydro-2H-benzimidazol-2-one;
5-chloro-{1-[4-(4-chlorophenyl)-5-methyl-4H-1,2,4-triazol-3-yl]piperidin-4-yl}-1,3-dihydro-2H-benzimidazol-2-one;
1-{1-[4-(4-chlorophenyl)-5-methyl-4H-1,2,4-triazol-3-yl]piperidin-4-yl}-5-fluoro-1,3-dihydro-2H-benzimidazol-2-one;
1-{1-[4-(4-chlorophenyl)-5-methyl-4H-1,2,4-triazol-3-yl]piperidin-4-yl}-1H-1,2,3-benzotriazole;
3-{1-[4-(4-chlorophenyl)-5-methyl-4H-1,2,4-triazol-3-yl]piperidin-4-yl}[1,3]oxazolo[4,5-b]pyridin-2(3H)-one;
3-{1-[4(4-chlorophenyl)-5-(2H-1,2,3-triazol-2-ylmethyl)-4H-1,2,4-triazol-3-yl]piperidin-4-yl}[1,3]oxazolo[4,5-b]pyridin-2(3H)-one;
1-[4-(4-chlorophenyl)-5-methyl-4H-1,2,4-triazol-3-yl]-4-(3-isopropyl-1,2,4-oxadiazol-5-yl)piperidine;
4-{1-[4-(4-chlorophenyl)-5-methyl-4H-1,2,4-triazol-3-yl]piperidin-4-yl}-2H-pyrido[3,2-b][1,4]oxazin-3(4H)-one;
1-[4-(4-chlorophenyl)-5-methyl-44-1,2,4-triazol-3-yl]-4-(5-isopropyl-1,2,4-oxadiazol-3-yl)piperidine;
1-[4-(4-chlorophenyl)-5-(2H-1,2,3-triazol-2-ylmethyl)-4H-1,2,4-triazol-3-yl]-4-(5-isopropyl-1,2,4-oxadiazol-3-yl)piperidine;
3-{1-[4-(4-Chlorophenyl)-5-methyl-4H-1,2,4-triazol-3-yl]piperidin-4-yl}[1,2,4]triazolo[4,3-b]pyridazine;
3-{1-[4-(4-Chlorophenyl)-5-methyl-4H-1,2,4-triazol-3-yl]piperidin-4-yl}-1,3-dihydro-2H-imidazo[4,5-b]pyridin-2-one;
3-{1-[4-(4-Chlorophenyl)-5-methyl-4H-1,2,4-triazol-3-yl]piperidin-4-yl}-3H-imidazo[4,5-b]pyridine;
3-{1-[4-(4-Chlorophenyl)-5-methyl-4H-1,2,4-triazol-3-yl]piperidin-4-yl}-3H-[1,2,3]triazolo[4,5-b]pyridine;
1-{1-[4-(4-Chlorophenyl)-5-methyl-4H-1,2,4-triazol-3-yl]piperidin-4-yl}-1H-benzimidazol-2-amine;
1-{1-[4-(4-Chlorophenyl)-5-methyl-4H-1,2,4-triazol-3-yl]piperidin-4-yl}-3-methyl-1,3-dihydro-2,1,3-benzothiadiazole 2,2-dioxide;
3-{1-[4-(4-Chlorophenyl)-5-methyl)-4H-1,2,4-triazol-3-yl]piperidin-4-yl}-6-fluoro-3H-[1,2,3]triazolo[4,5-b]pyridine;
3-{4-[4-(4-Chlorophenyl)-5-methyl-4H-1,2,4-triazol-3-yl]piperazin-1-yl}-1,2-benzisothiazole;
3-{4-[4-(4-Chlorophenyl)-5-methyl-4H-1,2,4-triazol-3-yl]piperazin-1-yl}-1,2-benzisothiazole 1,1-dioxide;
3-{4-[4-(4-Chlorophenyl)-5-(trifluoromethyl)-4H-1,2,4-triazol-3-yl]piperazin-1-yl}-1,2-benzisothiazole;
3-{4-[4-(4-Chlorophenyl)-5-(trifluoromethyl)-4H-1,2,4-triazol-3-yl]piperazin-1-yl}-1,2-benzisothiazole 1,1-dioxide;
3-{(3-endo)-8-[4-(4-Chlorophenyl)-5-methyl-4H-1,2,4-triazol-3-yl]-8-azabicyclo[3.2.1]oct-3-yl}-2-methyl-3H-imidazo[4,5-c]pyridine;
3-{4-[4-(4-Chlorophenyl)-5-methyl-4H-1,2,4-triazol-3-yl]piperidin-1-yl}-1,2-benzisothiazole;
3-{4-[4-(4-Chlorophenyl)-5-(methoxymethyl)-4H-1,2,4-triazol-3-yl]piperidin-1-yl}-1,2-benzisothiazole;
3-{4-[4-(4-Chlorophenyl)-5-methyl-4H-1,2,4-triazol-3-yl]piperidin-1-yl}isothiazolo[5,4-b]pyridine;
3-{4-[4-(4-Chlorophenyl)-5-methyl-4H-1,2,4-triazol-3-yl]piperidin-1-yl}-1,2-benzisothiazole 1,1-dioxide; and
3-{4-[4-(4-Chlorophenyl)-5-methyl-4H-1,2,4-triazol-3-yl]piperidin-1-yl}isoxazolo[4,5-b]pyridine;
or a pharmaceutically acceptable salt thereof.

30. A method of treating anxiety, cardiovascular disease, primary dysmenorrhea, secondary dysmenorrhea, endometriosis, emesis, intrauterine growth retardation, inflammation, mittlesmerchz, preclampsia, premature ejaculation, premature labor or Raynaud's disease in a mammal, the method comprising administering to the mammal in need of treatment thereof a therapeutically effective amount of a compound of claim 22.

31. The method of claim 30, wherein the disorder is primary dysmenorrhea or secondary dysmenorrhea.

32. The method of claim 30, wherein the disorder is anxiety.

33. A pharmaceutical composition comprising a compound of claim 22 or a pharmaceutically acceptable salt thereof, together with a pharmaceutically acceptable excipient, diluent or carrier.

34. A pharmaceutical composition comprising a combination of: (A) a compound according to claim 22; and (B) an additional pharmacologically active ingredient.

35. The composition of claim 34, wherein (B) is an oral contraceptive, PDEV inhibitor, COX inhibitor, NO-donor or L-arginine.

36. A method of treating primary dysmenorrhea or secondary dysmenorrhea, the method comprising administering to a patient in need of such treatment a combination of amounts of (A) and (B) according to claim 35, which are together effective in treating primary dysmenorrhea or secondary dysmenorrhea.
